(19) United States
(12) Patent Application Publication
(10) Pub. No.: US 2026/0335661 A1
(43) Pub. Date: Oct. 29, 2026

(54) POWER DELIVERY SYSTEMS FOR IMPLANTABLE MEDICAL DEVICES

(71) Applicant: Medtronic, Inc., Minneapolis, MN (US)

(72) Inventors: Gregory N. Nesseth, Forest Lake, MN (US); Jeffrey G. Falk, Ham Lake, MN (US); Paul T. Rothstein, Elk River, MN (US)

(21) Appl. No.: 19/650,150

(22) Filed: Apr. 16, 2026

Related U.S. Application Data
(60) Provisional application No. 63/794,243, filed on Apr. 24, 2025.

(51) Int. Cl. A61N 1/378; A61N 1/375

(52) U.S. Cl. CPC A61N 1/3787; A61N 1/37512; A61N 1/3756; A61N 1/37518

(57) ABSTRACT

Systems, devices, and methods for providing power to a medical device in a body of a patient are provided. In some embodiments, a system includes: an elongate shaft having a distal end that is configured to be positioned proximate to the medical device in the body of the patient; a hollow receptacle at the distal end of the elongate shaft, where the hollow receptacle is configured to receive at least a portion of the medical device; a first electrode configured to couple to a first portion of the medical device, where the first electrode is located on the hollow receptacle; a second electrode configured to couple to a second portion of the medical device; and an external power source configured to be electrically connected to the first and second electrodes to provide power to the medical device.

### CROSS-REFERENCE TO RELATED APPLICATION(S)

The present application claims the benefit of priority to U.S. Provisional Patent Application No. 63/794,243, filed Apr. 24, 2025, the disclosure of which is incorporated by reference herein in its entirety.

### TECHNICAL FIELD

The present technology generally relates to medical devices, and in particular, to power delivery systems for implantable medical devices.

### BACKGROUND

Various types of implantable medical devices have been developed for monitoring or treating one or more conditions of a patient. For example, a cardiac pacemaker can monitor a patient’s heart activity and provide therapeutic electrical stimulation to the heart via electrodes. The electrical stimulation provided by the cardiac pacemaker can include signals such as pacing pulses to address abnormal cardiac rhythms (e.g., bradycardia). Some types of cardiac pacemakers are implanted a distance from the heart and are coupled to one or more leads that extend intravascularly into the heart to position the electrodes in contact with cardiac tissue. However, the leads may be prone to fracture, which may result in unreliable or incorrect pacing, and may require replacement of the lead or even the entire pacemaker.

Some types of cardiac pacemakers are sized to be completely implanted within one of the chambers of the heart, and may include electrodes integrated with or attached to the device housing rather than leads. Such pacemakers can be less invasive than traditional pacemakers and can avoid complications associated with lead fracture. However, the relatively small size of such pacemakers may limit the types of power sources that can be incorporated into the device.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a pacing device implanted in the heart of a patient, in accordance with embodiments of the present technology.

FIG. 2 is a perspective view of a pacing device configured in accordance with embodiments of the present technology.

FIG. 3 is a side view of another pacing device configured in accordance with embodiments of the present technology.

FIG. 4 is a schematic block diagram illustrating electronic components of a pacing device configured in accordance with embodiments of the present technology.

FIG. 5 illustrates a system for providing power to a medical device in accordance with embodiments of the present technology.

FIGS. 6A–6D illustrate medical devices that are configured to couple to a power delivery system in accordance with embodiments of the present technology.

FIGS. 7A–7F illustrate hollow receptacles of a power delivery system in accordance with embodiments of the present technology.

FIGS. 8A and 8B illustrate capture elements of a power delivery system in accordance with embodiments of the present technology.

FIGS. 9A and 9B illustrate a power delivery system and medical device with polymeric seal in accordance with embodiments of the present technology.

### DETAILED DESCRIPTION

The present technology relates to a system for providing power to a medical device in a body of a patient. In some embodiments, the system includes an elongate shaft with a hollow receptacle at its distal end. The hollow receptacle can be configured to receive at least a portion of the medical device. A first electrode located on the hollow receptacle can be configured to couple to a first portion of the medical device, and a second electrode (e.g., located on the hollow receptacle or another component of the system) can be configured to couple to a second portion of the medical device. An external power source can be electrically connected to the first and second electrodes to deliver electrical energy to the medical device (e.g., for charging, directly powering device operations, communication, and/or diagnostics).

The present technology can provide many advantages compared to conventional implantable systems. Implantable medical devices generally have a finite life expectancy due to limitations in the power capacity of the internal power source of the device. In many cases, it may be impractical or impossible to retrieve the implanted device once the internal power source has been depleted. Instead, if a patient outlives the operational life of the medical device, an additional medical device may need to be implanted (e.g., in the vicinity of the initial medical device) to continue delivering therapy to the patient. However, it may be challenging to implant multiple devices in the patient, e.g., due to space constraints at the treatment site and/or potential adverse interactions between the two devices. Moreover, implantation of additional devices may be costly and time-consuming, and may require additional invasive surgical procedures. The present technology can address these and other challenge by providing a system for delivering power to an implanted medical device in situ within the patient’s body. The system can be configured to recharge the internal power source of the medical device, thereby extending the operational life of the implanted device (e.g., by months, years, or even indefinitely).

Although certain embodiments of the present technology are described herein in connection with medical devices implanted in a patient’s heart (e.g., pacing devices), this is not intended to be limiting, and the present technology may be applied to medical devices implanted in other locations of a patient’s body.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” “lower,” “left,” “right,” etc., can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I.Overview of Implantable Pacing Devices

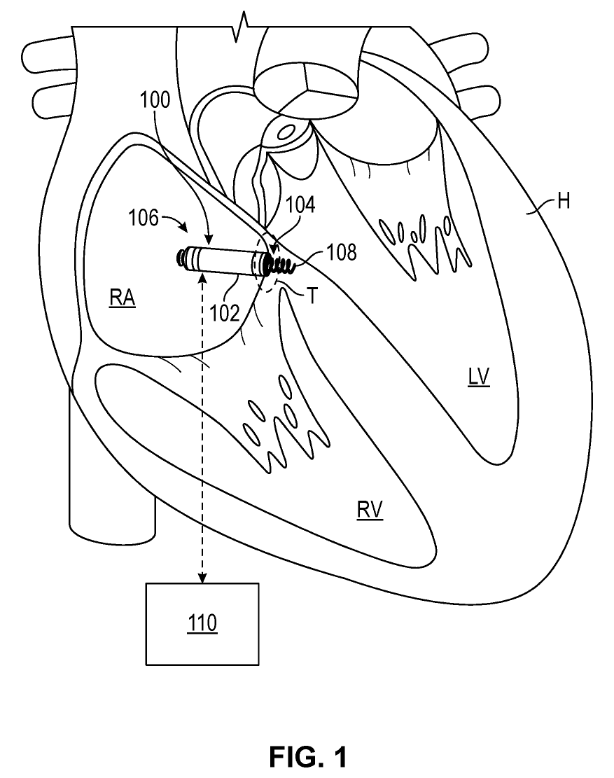

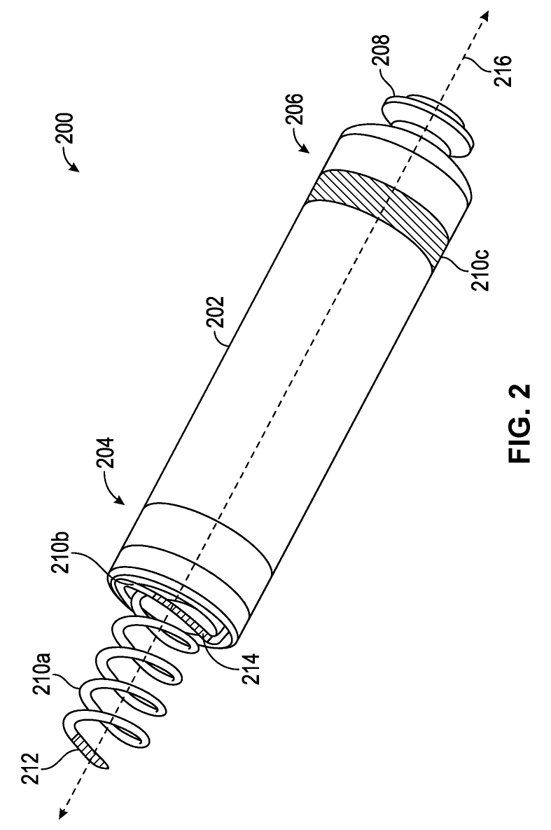

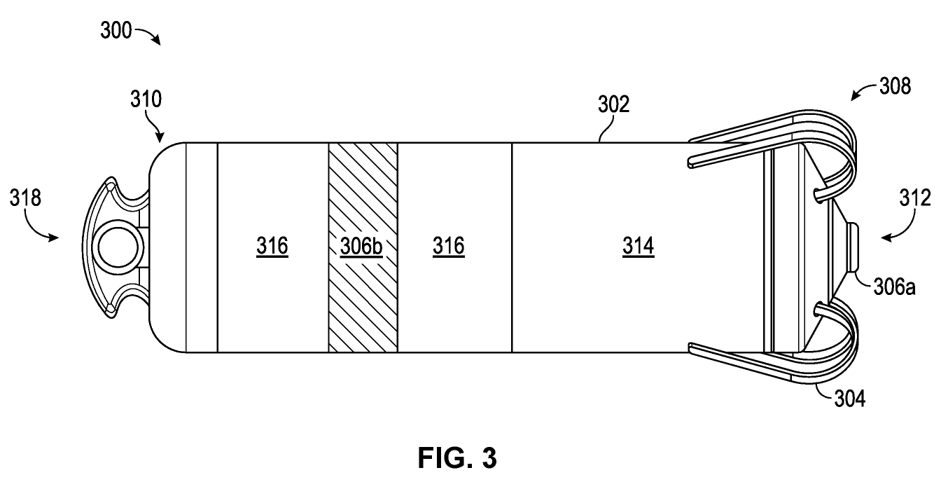

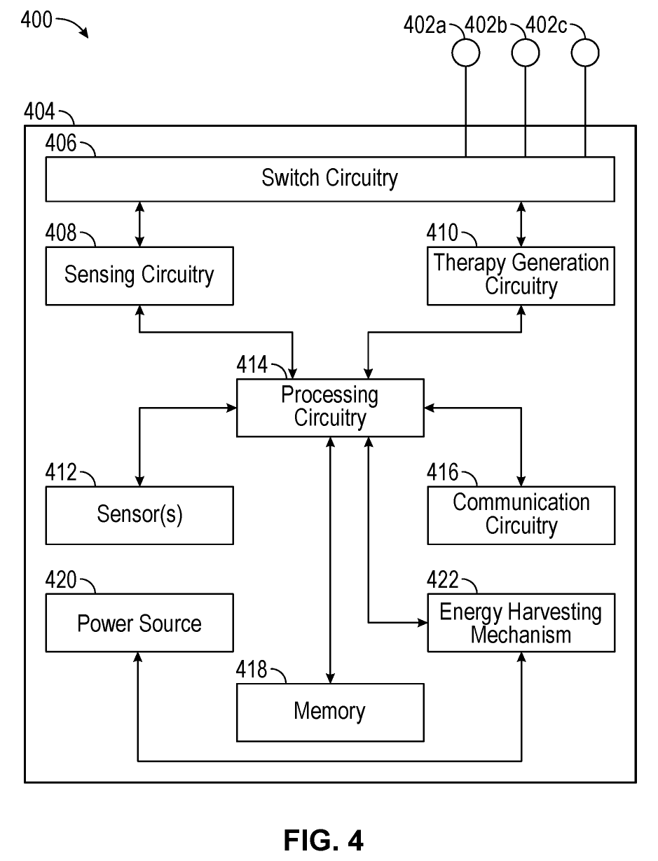

FIGS. 1–4 provide a general overview of implantable devices configured in accordance with embodiments of the present technology. Specifically, FIG. 1 illustrates a pacing device implanted in a patient’s heart, FIG. 2 illustrates an example configuration for a pacing device, FIG. 3 illustrates another example configuration for a pacing device, and FIG. 4 illustrates electronic components that can be included in a pacing device. Any of the features of the embodiments of FIGS. 1–4 can be combined with each other and/or with any of the other embodiments described herein.

Referring first to FIG. 1, which illustrates a pacing device 100 implanted in the heart H of a patient, the device 100 is configured to monitor activity of the heart H and provide electrical stimulation (e.g., pacing signals) to the heart H. In some embodiments, the device 100 is a leadless intracardiac pacemaker configured to be implanted entirely within a heart chamber, such as entirely within the right atrium (RA), entirely within the right ventricle (RV), entirely within the left atrium (LA), or entirely within the left ventricle (LV). The device 100 can be implanted at any of a variety of locations to sense and/or deliver therapy to any chamber or chambers of the heart H. For example, as shown in FIG. 1, the device 100 can be a right atrial intracardiac pacemaker that is implanted in the RA of the patient’s heart H in a target implant region T (e.g., the triangle of Koch). The target implant region T can lie between the bundle of His and the coronary sinus, and/or can be adjacent to the tricuspid valve. In other embodiments, the device 100 can instead be configured as a right ventricular intracardiac pacemaker that is implanted in the RV of the heart H, with the target implant region T lying along the endocardial wall at or near the apex of the RV.

The device 100 can include a housing 102 having a size and form factor suitable for transvenous delivery into the heart H via a catheter. In the illustrated embodiment, the housing 102 has an elongate shape extending from a distal portion 104 to a proximal portion 106. The housing 102 can have a generally cylindrical shape (e.g., pill-shaped or capsule-shaped), a generally prismatic shape (e.g., a rectangular prism), or any other suitable shape. The housing 102 can define an interior cavity that contains the electronic components of the device 100 (e.g., circuitry, power source, sensors).

The device 100 can include a fixation mechanism 108 to secure the device 100 to the tissue of the heart H. For example, the fixation mechanism 108 can include one or more fixation elements configured to penetrate into tissue, such as one or more tines, coils, barbs, etc. In the illustrated embodiment, the fixation mechanism 108 is coupled to and extends outwardly from the distal portion 104 of the housing 102. Accordingly, when the device 100 is implanted, the distal portion 104 can be positioned in contact with or in close proximity to the cardiac tissue, while the proximal portion 106 can be spaced apart from the cardiac tissue. In other embodiments, however, the fixation mechanism 108 can be located at a different portion of the device 100.

The device 100 also includes a plurality of electrodes configured to sense electrical activity of the heart H and/or deliver electrical therapy to the heart H. For example, the device 100 can include two, three, four, five, six, seven, eight, nine, ten, or more electrodes. Each electrode can be positioned at any suitable portion of the device 100, such as on or coupled to the housing 102 (e.g., the distal portion 104, the proximal portion 106, an intermediate location between the distal portion 104 and proximal portion 106), or on or coupled to the fixation mechanism 108. In some embodiments, the device 100 includes one or more electrodes (e.g., cathodes) that directly contact the cardiac tissue (e.g., of a single heart chamber or multiple heart chambers) to sense the activity thereof and/or deliver electrical therapy thereto. Such electrode(s) can be located at the distal portion 104 of the housing 102 and/or incorporated into the fixation mechanism 108, for example. The device 100 can also include at least one electrode (e.g., an anode and/or return electrode) that does not directly contact cardiac tissue. Such electrode(s) can be located at portions of the housing 102 that are spaced apart from cardiac tissue, such as the proximal portion 106. Optionally, a single electrode may serve as a cathode for certain operations, and may serve as an anode and/or return electrode for other operations.

In some embodiments, the device 100 is operably coupled to an external device 110 shown schematically) via bidirectional wireless communication, such as BLUETOOTH®, Wi-Fi, Medical Implant Communication Service (MICS), or other radiofrequency communication technique. The external device 110 can be a computing device or system that is located outside of the patient’s body, and can be used in a healthcare setting (e.g., in a clinic, hospital or other medical facility), at the patient’s home, or suitable combinations thereof. The external device 110 can be configured to control various operational parameters of the device 100, such as therapy parameters (e.g., pacing control parameters such as pacing interval), sensing parameters, power management parameters, etc. For instance, the external device 110 can transmit control signals to the device 100 to program one or more operational parameters of the device 100. Optionally, the external device 110 can display information relating to and/or received from the device 100, such as intracardiac electrogram (EGM) signals obtained by the device 100, motion sensor signals acquired by the device 100, operational parameters of the device 100, etc. In some embodiments, the external device 110 transmits information received from the device 100 to another computing device or system (e.g., a computer, laptop, workstation, mobile device, server, remote patient management system) for display, processing, and/or storage, using any suitable wired or wireless communication technique. The external device 110 can serve as a “programmer” that allows a physician, patient, or other individual to monitor and/or control the operations of the device 100.

Although FIG. 1 illustrates a single device 100, the present technology is also applicable to implantable systems including multiple devices 100 implanted at different locations in the heart H. For example, an implantable system can include a first device 100 in the RA and a second device 100 in the RV. In such embodiments, each device 100 can independently have any of the features described herein.

FIG. 2 is a perspective view of a pacing device 200 configured in accordance with embodiments of the present technology. The device 200 is configured to be implanted within a chamber of a heart of the patient to monitor activity of the heart and/or provide electrical therapy (e.g., pacing therapy) to the heart. The device 200 includes a housing 202 having a size and form factor that allows the device 200 to be entirely implanted within a single chamber of the patient’s heart. In the illustrated embodiment, the housing 202 has an elongate shape (e.g., a generally cylindrical shape, a generally prismatic shape) extending between a distal end 204 and proximal end 206. The housing 202 can define a hermetically sealed internal cavity for housing the electronic components of the device 200. The housing 202 can also include an attachment mechanism 208 (e.g., at the proximal end 206) configured to temporarily engage with a delivery tool during implantation and/or extraction of the device 200.

The housing 202 can be formed partially or entirely from a conductive material, such as titanium or titanium alloy, stainless steel, MP35N (a non-magnetic nickel-cobalt-chromium-molybdenum alloy), a platinum alloy, or other biocompatible metal or metal alloy, or other suitable conductive material. Alternatively or in combination, the housing 202 can be formed partially or entirely from a nonconductive (e.g., insulative) material, such as ceramic, glass, sapphire, silicone, polyurethane, epoxy, acetyl co-polymer plastics, polyether ether ketone (PEEK), a liquid crystal polymer, other biocompatible polymer, or other suitable nonconductive material.

The device 200 can include a plurality of electrodes 210a–210c configured to sense electrical activity of the heart and/or deliver electrical stimulation to the heart. In the illustrated embodiment, for example, the device 200 includes a first electrode 210a and a second electrode 210b at or proximate to the distal end 204 of the housing 202, and a third electrode 210c on the housing 202. The first and second electrodes 210a, 210b can be configured as cathode electrodes that directly contact cardiac tissue, e.g., a distal end of the first electrode 210a can be configured to rest within a ventricular myocardium of the patient, and the second electrode 210b can be configured to contact an atrial endocardium of the patient. The third electrode 210c can be configured as an anode and/or return electrode that does not directly contact cardiac tissue.

As shown in FIG. 2, the first electrode 210a can be an elongate structure that extends from the distal end 204 of the housing 202 to penetrate through the wall tissue of a first heart chamber (e.g., the chamber in which the device 200 is implanted) into wall tissue of a second, different heart chamber. For example, in some embodiments, the device 200 is implanted in the RA with the distal end 204 oriented toward the LV (e.g., similar to the arrangement of the device 100 in FIG. 1), and the first electrode 210a extends through the wall tissue of the RA and into the wall tissue of the LV. In the illustrated embodiment, the first electrode 210a is configured as a coil (e.g., a helical and/or spiral coil), while in other embodiments, the first electrode 210a can have a different form factor (e.g., an elongate dart, barb, tine, or other tissue penetrating element). The first electrode 210a can include a proximal end that is coupled to the distal end 204 of the housing 202, and a free distal end that is not attached to the housing 202. The distal end of the first electrode 210a can have a conical, hemi-spherical, or slanted edge distal tip with a narrow tip diameter (e.g., less than 1 mm) for penetrating into and through tissue layers. In some embodiments, the distal end of the first electrode 210a can have a sharpened or angular tip, and/or sharpened or beveled edges, but the degree of sharpness can be constrained to avoid a cutting action that could lead to lateral displacement of the distal end of the first electrode 210a and undesired tissue trauma.

The second electrode 210b can be a structure that extends from the distal end 204 of the housing 202 to contact the wall tissue of the first heart chamber without penetrating the wall tissue. The second electrode 210b can be located proximal to the first electrode 210a. The second electrode 210b can be configured as a coil (e.g., a partial helical and/or spiral coil that does not form a full turn), loop, button, pad, or any other suitable form factor. The second electrode 210b can include a proximal end that is coupled to the distal end 204 of the housing 202, and a distal end that may or may not be coupled to the housing 202. In some embodiments, the second electrode 210b is configured to flexibly maintain contact with wall tissue of the heart chamber in which the device 200 is implanted, (e.g., the RA endocardium), despite variations in the tissue surface and/or in the distance between the distal end 204 of the housing 202 and the tissue surface, which may occur as the wall tissue moves during the cardiac cycle. Accordingly, the second electrode 210b can be flexible and/or have spring-like properties, e.g., the second electrode 210b can have a spring bias that urges at least a portion of the second electrode 210b away from the distal end 204 of the housing 202 and toward the wall tissue of the heart chamber to maintain consistent contact.

The first and second electrodes 210a, 210b can each be formed of an electrically conductive material, such as titanium, platinum, iridium, tantalum, or alloys thereof. The first electrode 210a can include one or more insulative coatings (e.g., parylene, polyurethane, silicone, epoxy) that reduce the electrically conductive surface area of the first electrode 210a to define a first electrically active region 212 (e.g., at or near the distal end of the first electrode 210a). The second electrode 210b can include one or more insulative coatings (e.g., parylene, polyurethane, silicone, epoxy) that reduce the electrically conductive surface area of the second electrode 210b to define a second electrically active region 214 (e.g., at an intermediate region between the proximal and distal ends of the second electrode 210b). This approach can increase the electrical impedance of the first and second electrodes 210a, 210b, and thereby reduce the current delivered during a pacing pulse, which can conserve the power used by the device 200. In some embodiments, the first and second electrodes 210a, 210b include an electrically conductive material coating (e.g., TiN) on the first and second electrically active regions 212, 214, respectively, to define the active regions. The first and second electrodes 210a, 210b can be made of the same materials, or can be made of different materials.

All, substantially all, or a portion of the housing 202 can serve as a third electrode 210c (e.g., an anode and/or return electrode) during pacing and/or sensing. In some embodiments, the third electrode 210c partially or fully circumscribes a portion of the housing 202 at or near the proximal end 206. Although FIG. 2 illustrates the third electrode 210c as a singular band, in other embodiments, the third electrode 210c can include multiple segments spaced a distance apart along a longitudinal axis 216 of the housing 202 and/or around a perimeter of the housing 202. Additionally, the third electrode 210c can also be located at other positions along the housing 202, e.g., located at or near the distal end 204 or at other positions along the longitudinal axis 216.

In embodiments where the housing 202 is formed from a conductive material, one or more portions of the housing 202 can be electrically insulated by a nonconductive material, such as a coating of parylene, polyurethane, silicone, epoxy or other biocompatible polymer, or other suitable material. For the portions of the housing 202 without the nonconductive material, one or more discrete areas of the housing 202 with conductive material can be exposed to define the third electrode 210c. In embodiments where the housing 202 is formed from a nonconductive material, a conductive material can be applied to one or more discrete areas of the housing 202 to form the third electrode 210c. Optionally, the third electrode 210c can be a discrete component (e.g., a ring electrode) that is coupled to the housing 202.

The electrodes 210a–210c can be used to sense electrical activity of one or more heart chambers and/or to deliver electrical stimulation to one or more heart chambers. For example, the first electrode 210a can be paired with the second electrode 210b or the third electrode 210c to for sensing ventricular signals and delivering ventricular pacing pulses. As another example, the second electrode 210b can be paired with the first electrode 210a or the third electrode 210c for sensing atrial signals and delivering pacing pulses to the atrial myocardium. In a further example, the third electrode 210c can be paired at different times with both the first electrode 210a and the second electrode 210b for either ventricular or atrial functionality, respectively. As yet another example, the first electrode 210a and the second electrode 210b can be paired with each other with different polarities for atrial and ventricular functionality.

In some embodiments, the second electrode 210b is configured as an atrial cathode electrode for delivering pacing pulses to the atrial tissue at a target implant region in combination with the third electrode 210c. The second electrode 210b and the third electrode 210c can also be used to sense atrial P-waves for use in controlling atrial pacing pulses (e.g., delivered in the absence of a sensed P-wave) and for controlling atrial-synchronized ventricular pacing pulses delivered using the first electrode 210a as a cathode and the third electrode 210c as the return anode. The configuration of the electrodes 210a–210c illustrated in FIG. 2 allows the device 200 to sense cardiac signals from and/or deliver cardiac pacing to one or more chambers of the heart. For example, the present technology can facilitate the delivery of A-V synchronous pacing using a single device 200 implanted within a single heart chamber (e.g., the RA).

The device 200 can include a fixation mechanism 218 configured to fix the device 200 to cardiac tissue at a target implant region (e.g., the triangle of Koch). In the illustrated embodiment, the first electrode 210a and/or second electrode 210b at the distal end 204 of the housing 202 can serve as the fixation mechanism 218. In other embodiments, the fixation mechanism 218 can be a different component than the first electrode 210a and/or the second electrode 210b, such one or more separate barbs, tines, coils, darts, etc.

FIG. 3 is a side view of another pacing device 300 configured in accordance with embodiments of the present technology. The device 300 is configured to be implanted within a chamber of a heart of a patient to monitor activity of the heart and/or to provide electrical therapy to the heart. In the embodiment shown in FIG. 3, the device 300 includes a housing 302, a plurality of fixation tines 304, a first electrode 306a, and a second electrode 306b.

The housing 302 can have a size and form factor that allows the device 300 to be entirely implanted within a chamber of a heart of a patient. For example, as shown in FIG. 3, the housing 302 has a generally cylindrical (e.g., pill-shaped or capsule-shaped), elongate form factor extending between a distal end 308 and a proximal end 310. The housing 302 contains electronic components of the device 300, and can be hermetically or near-hermetically sealed to prevent fluid ingress into the housing 302. The materials used to form the housing 302 can include any of the conductive and nonconductive materials described above with respect to FIG. 2.

The device 300 can include a fixation mechanism configured to fix the device 300 to cardiac tissue at a target implant region (e.g., the endocardial wall near the apex of the RV). In the illustrated embodiment, the device 300 includes a plurality of fixation tines 304 extending from the distal end 308 of the housing 302 and configured to engage with cardiac tissue to secure the housing 302 at a fixed position within the chamber of the heart. The fixation tines 304 can be configured to anchor the housing 302 to the cardiac tissue such that the device 300 moves along with the cardiac tissue during cardiac contractions. The device 300 can include any suitable number of fixation tines 304, such as one, two, three, four, five, or more fixation tines 304. The fixation tines 304 can be fabricated from any suitable material, such as a shape memory material (e.g., Nitinol). Alternatively or in combination, the device 300 can be fixed to cardiac tissue using other types of fixation mechanisms, such as, but not limited to, barbs, coils, darts, and the like.

Optionally, the device 300 can include an attachment mechanism configured to temporarily couple the device 300 to a delivery tool, e.g., for delivery and/or extraction of the device 300. In the illustrated embodiment, for example, the proximal end 310 includes a flange 318 that defines an opening. The flange 318 can be attached to a tether (e.g., by threading the tether through the opening) that extends through an elongate shaft (e.g., a catheter) to implant or extract the device 300.

In some embodiments, the device 300 is configured to sense electrical activity of the heart and/or deliver electrical stimulation to the heart via the first electrode 306a and second electrode 306b (collectively, “electrodes 306”). The first electrode 306a can serve as a cathode configured to electrically contact cardiac tissue and deliver pacing pulses thereto, and the second electrode 306b can serve as an anode and/or a return electrode. Optionally, the device 300 can be equipped with multiple cathode electrodes. Such multiple cathode electrodes can be configured to electrically contact and deliver pacing pulses to cardiac tissue of a single heart chamber, or cardiac tissue of multiple heart chambers. In some such embodiments, the multiple cathode electrodes are configured to electrically contact and deliver pacing pulses to cardiac tissue of different heart chambers. For example, one cathode electrode can be configured to electrically contact and deliver pacing pulses to atrial tissue, and another cathode electrode may be configured to electrically contact and deliver pacing pulses to ventricular tissue.

The electrodes 306 can be configured in many different ways. For example, one or both of the electrodes 306 can be discrete components that are mechanically coupled to the housing 302. As another example, one or both of the electrodes 306 can be defined by an outer portion of the housing 302 that is electrically conductive. The electrodes 306 can be electrically isolated from each other. In some embodiments, a portion of the housing 302 is covered by or formed from an insulative material to isolate the electrodes 306 from each other and/or to provide a desired size and shape for one or both of the electrodes 306. The electrodes 306 can be electrically coupled to at least some of the internal electronic components of the device 300 within the housing 302 (e.g., sensing circuitry, electrical stimulation circuitry, or both).

In the illustrated embodiment, the first electrode 306a is located at the distal end 308 of the housing 302. The first electrode 306a may be referred to as a tip electrode, and the fixation tines 304 can be configured to anchor the device 300 to cardiac tissue such that the first electrode 306a maintains contact with the cardiac tissue. In some examples, the housing 302 includes an end cap 312 at the distal end 308, and the end cap 312 includes a feedthrough assembly to electrically couple the first electrode 306a to the electronics within the housing 302, while electrically isolating the first electrode 306a from the remaining portions of the housing 302, e.g., including the second electrode 306b and/or other conductive portions of the housing 302

The second electrode 306b can be located on the housing 302 away from (e.g., proximal to) the first electrode 306a. As shown in FIG. 3, the housing 302 includes a first portion 314 and a second portion 316, with the first portion 314 being located proximal to the end cap 312, and the second portion 316 being located proximal to the first portion 314. The second portion 316 can optionally define at least part of a power source case that houses a power source (e.g., a battery) of the pacing device 300. In some embodiments, the second electrode 306b is located on the second portion 316, while in other embodiments, the second electrode 306b is located on the first portion 314.

In some embodiments, the second electrode 306b is a conductive portion of the housing 302 (e.g., an annular portion of the housing 302 that is made partially or entirely from a conductive material). Additionally or alternatively, the second electrode 306b can be a conductive material that is coated onto the material of the housing 302, or a discrete component (e.g., a ring electrode) that is coupled to the housing 302. The remaining portions of the housing 302 can include or be coated with an insulative material so that the second electrode 306b is electrically isolated from the rest of the housing 302 and/or from the first electrodes 306a.

FIG. 4 is a schematic block diagram illustrating electronic components of a pacing device 400 configured in accordance with embodiments of the present technology. Any of the electronic components shown in FIG. 4 can be incorporated into any of the embodiments of implantable devices described herein, such as the device 100 of FIG. 1, the device 200 of FIG. 2, or the device 300 of FIG. 3.

As shown in FIG. 4, the device 400 includes a plurality of electrodes 402a–402c that are electrically coupled to components within a housing 404 of the device 400. Although the device 400 is illustrated and described herein as having three electrodes 402a–402c (e.g., similar to the device 200 of FIG. 2), in other embodiments, the device 400 can be modified to include a different number of electrodes, such as two electrodes (e.g., similar to the device 300 of FIG. 3) or any other suitable number of electrodes.

At least some of the electrodes 402a–402c can be configured to contact tissue of one or more heart chambers, as described elsewhere herein. For example, as discussed above with respect to FIG. 2, the first electrode 402a can be configured to electrically contact and deliver electrical signals to tissue of a first heart chamber (e.g., ventricular tissue), and the second electrode 402b can be configured to electrically contact and deliver electrical signals to tissue of a second, different heart chamber (e.g., atrial tissue). The third electrode 402c can be an anode and/or return electrode that does not electrically contact heart tissue. Optionally, either the first electrode 402a or the second electrode 402b can be omitted, or the device 400 can include additional electrodes that electrically contact and deliver electrical signals to tissue of a heart chamber (e.g., the first heart chamber, the second heart chamber, or another heart chamber).

The device 400 includes a plurality of electronic components within the housing 404, such as switch circuitry 406, sensing circuitry 408, therapy generation circuitry 410, one or more sensors 412, processing circuitry 414, communication circuitry 416, memory 418, and/or a power source 420. The various circuitry can be or include programmable or fixed function circuitry configured to perform the operations described herein. One or more of the components of the device 400 shown in FIG. 4 can be part of an electronics assembly. For example, one or more of the switch circuitry 406, sensing circuitry 408, therapy generation circuitry 410, sensor(s) 412, processing circuitry 414, communication circuitry 416, and/or memory 418 can be mounted on a circuit board of an electronics assembly of the device 400.

The switch circuitry 406 can include one or more switches (e.g., a switch matrix, switch arrays, or other collection of switches), multiplexers, transistors, and/or other electrical circuitry. The switch circuitry 406 can selectively couple one or more of the electrodes 402a–402c to other components of the device 400 (e.g., the sensing circuitry 408 and/or the therapy generation circuitry 410). The subset of the electrodes 402a–402c to be used can depend on the particular operation of the device 400 that is being performed, such as whether the device 400 is sensing or delivering therapy, the locations of the heart being monitored or treated, etc. In some embodiments, the processing circuitry 414 determines which subset of the electrodes 402a–402c should be used for a particular operation, and controls the switch circuitry 406 to selectively couple those electrodes to the appropriate components of the device 400.

The sensing circuitry 408 can monitor signals from at least one of electrodes 402a–402c to monitor electrical activity of the heart, impedance, and/or other electrical phenomena. Sensing can be performed to determine heart rates and/or heart rate variability; and/or to detect ventricular dyssynchrony, arrhythmias (e.g., tachyarrhythmias), and/or other electrical signals. The sensing circuitry 408 can include filters, amplifiers, analog-to-digital converters, and/or other circuitry configured to sense cardiac electrical signals via one or more of the electrodes 402a–402c.

In some embodiments, the switch circuitry 406 as controlled by the processing circuitry 414 selectively couples the sensing circuitry 408 to selected combinations of the electrodes 402a–402c, e.g., to selectively sense the electrical activity of one or more chambers of the heart. For example, the switch circuitry 406 can couple each of the first electrode 402a and the second electrode 402b (in combination with the third electrode 402c) to respective sensing channels provided by the sensing circuitry 408 to sense electrical signals from the cardiac tissues in electrical contact with the first electrode 402a (e.g., ventricular tissue) and the second electrodes 402b (e.g., atrial tissue), respectively. In some embodiments, the sensing circuitry 408 is configured to detect events, (e.g., depolarizations) within the cardiac electrical signals, and to provide indications thereof to the processing circuitry 414. In this manner, the processing circuitry 414 can determine the timing of atrial and/or ventricular depolarizations, and can control the delivery of cardiac pacing (e.g., AV synchronized cardiac pacing) based thereon.

The therapy generation circuitry 410 can generate electrical stimulation signals, such as cardiac pacing pulses. The therapy generation circuitry 410 can be electrically coupled to one or more of the electrodes 402a–402c to deliver pulses to a portion of cardiac muscle within the heart via one or more of the electrodes 402a–402c. In some embodiments, the therapy generation circuitry 410 delivers pacing stimulation in the form of electrical pulses. The therapy generation circuitry 410 can include charging circuitry, and one or more charge storage devices (e.g., capacitors). Optionally, the therapy generation circuitry 410 can include switches and/or other circuitry to control when the charge storage devices are discharged to the electrodes 402a–402c.

The switch circuitry 406 as controlled by the processing circuitry 414 can direct electrical stimulation signals from the therapy generation circuitry 410 to a selected combination of the electrodes 402a–402c having selected polarities, e.g., to selectively deliver pacing pulses to the RA, RV, LV, and/or the interventricular septum of the heart. For example, in order to pace one or both of the ventricles, the switch circuitry 406 can electrically couple the first electrode 402a (e.g., which contacts wall tissue of a ventricle or the intraventricular septum) to the therapy generation circuitry 410 as a cathode, and to one or both of the second electrode 402b or the third electrode 402c to the therapy generation circuitry 410 as an anode. As another example, in order to pace the RA, the switch circuitry 406 can couple the second electrode 402b (e.g., which contacts the RA endocardium) to the therapy generation circuitry 410 as a cathode, and to one or both of the first electrode 402a or the third electrode 402c to the therapy generation circuitry 410 as an anode.

The processing circuitry 414 can include one or more processors, such as a microprocessor, a controller, a digital signal processor (DSP), an application specific integrated circuit (ASIC), a field-programmable gate array (FPGA), or equivalent discrete or analog logic circuitry. In some embodiments, the processing circuitry 414 can include multiple components, such as any combination of one or more microprocessors, controllers, DSPs, ASICs, and/or FPGAs, as well as other discrete or integrated logic circuitry. The functions attributed to the processing circuitry 414 herein may be embodied as software, firmware, hardware, or any combination thereof.

The processing circuitry 414 can control the therapy generation circuitry 410 to deliver stimulation therapy to a patient’s heart according to therapy parameters, which can be stored in the memory 418. For example, the processing circuitry 414 can control the therapy generation circuitry 410 to deliver electrical pulses with the amplitudes, pulse widths, rates, frequencies, and/or electrode polarities specified by the therapy parameters. In this manner, the therapy generation circuitry 410 can deliver pacing pulses to the heart via one or more of the electrodes 402a–402c. The device 400 can use any combination of the electrodes 402a–402c to deliver therapy and/or detect electrical signals from the patient.

The memory 418 (e.g., a data storage device or other non-transitory medium) can store computer-readable instructions that, when executed by the processing circuitry 414, cause the device 400 to perform the various operations described herein. The memory 418 can include any volatile, non-volatile, magnetic, optical, or electrical media, such as a random-access memory (RAM), read only memory (ROM), non-volatile RAM (NVRAM), electrically-erasable programmable ROM (EEPROM), flash memory, or any other digital or analog media.

The sensor(s) 412 can include one or more sensing elements that transduce patient physiological activity to an electrical signal to sense values of a respective patient parameter. Sensor(s) 412 can include one or more motion sensors, optical sensors, chemical sensors, temperature sensors, pressure sensors, and/or any other types of sensors. The sensor(s) 412 can output patient parameter values to the processing circuitry 414 that can be used as feedback to control sensing and/or delivery of therapy by the device 400.

For example, the sensor(s) 412 can include at least one motion sensor, such as one or more inertial measurement units (IMUs), accelerometers, gyroscopes, electrical or magnetic field sensors, and/or other devices capable of detecting motion and/or the position of the device 400. The motion of the device 400 detected by the motion sensor may be indicative of cardiac events (e.g., paced activation of the ventricles), blood flow through the heart, patient posture, patient activity, and/or noise. The processing circuitry 414 can control and/or monitor the motion data produced by the motion sensor to identify one or more features of the cardiac contraction within the signal (e.g., on a beat-by-beat basis or otherwise) to facilitate delivery of therapy (e.g., delivery of ventricular pacing pulses in an atrial-synchronized manner). Optionally, the processing circuitry 414 can use the motion data to detect a current activity level of the patient, which can be used for rate-responsive pacing of the patient’s heart.

The communication circuitry 416 is configured to allow the device 400 to wirelessly communicate with another device, such as a device external to the patient’s body (e.g., the external device 110 of FIG. 1) and/or another device under the control of the processing circuitry 414. For instance, the processing circuitry 414 can receive updates to operational parameters from the other device, and/or can provide collected data, (e.g., sensed heart activity and/or other patient parameters) to the other device via the communication circuitry 416. The communication circuitry 416 can use radiofrequency (RF) communication techniques (e.g., via an antenna) and/or any other suitable communication modality.

The power source 420 delivers operating power to various components of the device 400. The power source 420 can include one or more batteries, each of which can independently be rechargeable or non-rechargeable. Recharging of the power source 420 can be accomplished through proximal inductive interaction between an external charger and an inductive charging coil within the device 400. Alternatively or in combination, recharging of the power source 420 can be accomplished using an energy harvesting mechanism 422 of the device 400. Optionally, recharging of the power source 420 can be accomplished using a power delivery system as described in Section II below.

The components of the device 400 illustrated in FIG. 4 can be modified in many different ways. For example, any of the components shown in FIG. 4 can be combined with each other, e.g., the switch circuitry 406 can be incorporated into the sensing circuitry 408 and/or the therapy generation circuitry 410. Any of the components shown in FIG. 4 can be divided into smaller subcomponents. Some of the components in FIG. 4 are optional and may be omitted (e.g., the switch circuitry 406 and/or sensor(s) 412). The device 400 can also include additional components not shown in FIG. 4. For example, the device 400 can include power management circuitry coupled to the power source 420 to allow the processing circuitry 414 to monitor the status of the power source 420 (e.g., charge level, charging rate, net power into and/or out of the power source 420, remaining battery life).

The components of the device 400 shown in FIG. 4 represent functionality that can be included in any of the devices of the present technology. The components illustrated in FIG. 4 can include any discrete and/or integrated electronic circuit components that implement analog and/or digital circuits capable of producing the functions attributed to the components herein. For example, the components can include analog circuits, such as amplification circuits, filtering circuits, and/or other signal conditioning circuits. The components can also include digital circuits, such as combinational or sequential logic circuits, memory devices, and the like. The functions attributed to the components of FIG. 4 may be embodied as one or more processors, hardware, firmware, software, or any combination thereof. The depiction of different features as separate blocks in FIG. 4 is intended to highlight different functional aspects, and does not necessarily imply that such components must be realized by separate hardware or software components. Rather, functionality associated with one or more components may be performed by separate hardware or software components, or integrated within common or separate hardware or software components. For example, although illustrated as separate functional components in FIG. 4, some or all of the functionality attributed to the switch circuitry 406, sensing circuitry 408, therapy generation circuitry 410, sensor(s) 412, and/or communication circuitry 416 can alternatively or additionally be implemented by the processing circuitry 414, or vice-versa.

### II.Power Delivery Systems

In some embodiments, the present technology provides systems for providing power to an implantable device within a patient’s body. The power capacity of a power source of an implantable device may be limited due to size constraints, such as if the device is implanted within a small space within the patient’s body (e.g., within a single heart chamber) and/or to avoid the device interfering with normal physiological function, as well as safety considerations. In some instances, a powered implanted device (e.g., a leadless pacemaker) may be left in place after its power source has been depleted due to lack of a mechanism to recharge or retrieve the device, which can create complications for the patient (e.g., the depleted device may obstruct the placement of additional devices and/or other interventions at the implantation site). To prolong the usable life of such implantable devices, a power delivery system can be used to provide energy to the implantable device in situ to charge the power source.

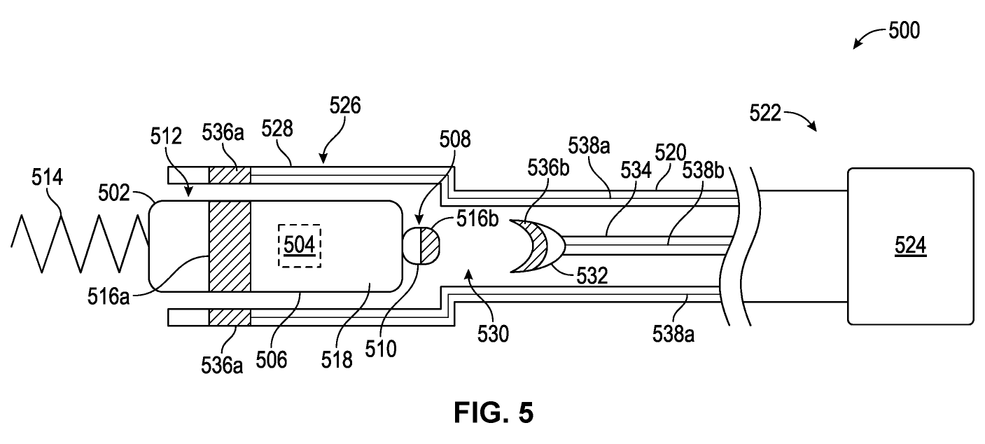

FIG. 5 illustrates a partially schematic cross-sectional view of a system 500 for providing power to a medical device 502, in accordance with embodiments of the present technology. The system 500 can be configured to deliver electrical energy (e.g., power) to the medical device 502 while the medical device 502 is implanted (e.g., in a heart of a patient). The system 500 can deliver electrical energy via one or more electrodes (e.g., first and second electrodes) configured to couple to (e.g., physically and/or electrically contact) the medical device 502. The system can further include one or more components that receive and/or capture the medical device 502 to bring the electrode(s) into alignment with the medical device to establish the coupling and allow delivery of electrical energy.

The medical device 502 can be any device that is configured to perform diagnostics and/or deliver therapy when implanted within a patient (e.g., in a heart of the patient). For instance, the medical device 502 may be a pacing device (e.g., a leadless pacemaker) as described in Section I above. However, other types of implantable devices may also be used as the medical device 502, such as monitoring devices, neuromodulation devices, etc.

The medical device 502 has a housing 506 configured to contain and shield internal components of the medical device 502 from the surrounding environment while implanted, such as an internal power source 504 (e.g., battery) and/or any of the other components described in Section I above. The housing 506 may be identical or generally similar to, for example, the housing 102 of FIG. 1, the housing 202 of FIG. 2, the housing 302 of FIG. 3, and/or the housing 404 of FIG. 4. The housing 506 can also be configured to couple to the system 500 to allow the system 500 to receive at least a portion of the implanted medical device 502 for delivering electrical energy to the medical device 502. For example, the housing 506 can include a proximal end 508 that optionally includes an attachment mechanism 510 (e.g., a protrusion, flange, opening) configured to couple to one or more components of the system 500 so that the medical device 502 can be received and/or powered by the system 500, as described in further detail below. The housing 506 can include a distal end 512 that optionally includes a fixation mechanism 514 (e.g., one or more coils, barbs, tines) configured to affix the medical device 502 to tissue at the implantation site. Accordingly, when implanted, the distal end 512 of the housing 506 may be positioned proximate to the tissue, while the proximal end 508 of the housing 506 is spaced away from the tissue and exposed for capture by the system 500. In other embodiments, however, the attachment mechanism 510 and fixation mechanism 514 can be located at different portions of the housing 506, or may be omitted altogether.

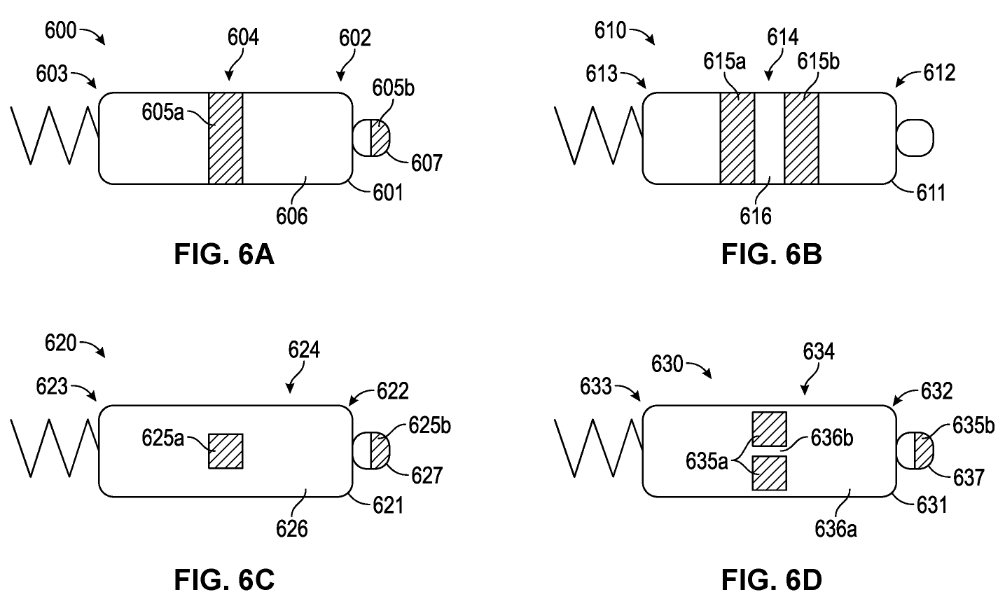

In some embodiments, the housing 506 includes one or more portions that are configured to electrically couple to the system 500. In the example embodiment shown in FIG. 5, the one or more portions of the housing 506 include a first portion 516a that is located between the proximal end 508 and the distal end 512 of the housing 506, and a second portion 516b that is located at the proximal end 508 of the housing 506 (e.g., on the attachment mechanism 510) (collectively, “portions 516”). In other embodiments, however, the housing 506 can include a different number of portions 516 (e.g., one, three, four, five, or more portions 516) and/or the portions 516 can be located at different portions of the housing 506. Example configurations of the portions 516 of the housing 506 are described below with respect to FIGS. 6A–6D.

The one or more portions 516 can be made from a conductive material such as a conductive metal (e.g., titanium or titanium alloy, stainless steel, MP35N, a platinum alloy), a conductive polymer, or any combination thereof. Each of the one or more portions 516 can be individually electrically addressable by electrically insulating each of the one or more portions 516 from each other. In some embodiments, the one or more portions 516 can be physically spaced apart from each other and separated by an insulating region 518 composed of a non-conductive (e.g., insulative) material (e.g., ceramic, glass, sapphire, silicone, polyurethane, epoxy, acetyl co-polymer plastics, PEEK, a liquid crystal polymer, other biocompatible polymer), so that the insulating region 518 electrically isolates the individual portions 516 from each other.

The one or more portions 516 can be configured to electrically couple to one or more internal components of the medical device 502, such as the power source 504 and/or electrical circuitry (not shown) associated with the power source 504. For instance, the medical device 502 may include wires, pins, contacts, and/or other electrical connectors extending between the portions 516 and the power source 504 and/or other electrical circuitry of the medical device 502. In some embodiments, electrical energy received by the one or more portions 516 from the system 500 charges the power source 504, as described further below. For instance, the first portion 516a may be coupled to a positive terminal of the power source 504 and the second portion 516b may be coupled to a negative terminal of the power source 504, or vice-versa.

The system 500 can be configured to deliver electrical energy to the medical device 502 (e.g., via the one or more portions 516 of the housing 506) using one or more electrodes on an elongate shaft 520. The elongate shaft 520 can be a catheter, a sheath, or other component configured to navigate the interior of the patient’s body (e.g., through the vasculature and/or other body lumens). The elongate shaft 520 can include a proximal end 522 that connects to an external power source 524 configured to provide electrical energy, and a distal end 526 that is configured to be introduced to a target site within the body of the patient proximate to the medical device 502. The distal end 526 can include a hollow receptacle 528 configured to receive at least a portion (e.g., a proximal portion) of the medical device 502. In some embodiments, the elongate shaft 520 includes a lumen 530 extending from the proximal end 522 to the distal end 526 to receive one or more components of the system 500. In some embodiments, the lumen 530 extends distally through the hollow receptacle 528.

The hollow receptacle 528 of the elongate shaft 520 is configured to receive and hold the medical device 502 and deliver electrical energy thereto (e.g., via one or more electrodes on the hollow receptacle 528, as discussed further below). The hollow receptacle 528, as shown in FIG. 5, can be configured to surround at least a portion of the medical device 502 when the system 500 receives the medical device 502. In some embodiments, the hollow receptacle 528 takes the form of a cup, a sheath, or other component suitable for covering a portion of the medical device 502.

The hollow receptacle 528 can be sized and shaped to accommodate the medical device 502, e.g., the size and shape of the hollow receptacle 528 can be based at least in part on the size and shape of the portion of the medical 502 device to be received. For instance, the inner diameter and/or width of the hollow receptacle 528 can be greater than or equal to the outer diameter and/or width of the received portion of the medical device 502; the length of the hollow receptacle 528 can be at least 10%, 20%, 30%, 40%, 50%, 60%, 70%, 80%, 90%, or 100% of the length of the medical device 502); both components can have substantially the same cross-sectional shape; etc.

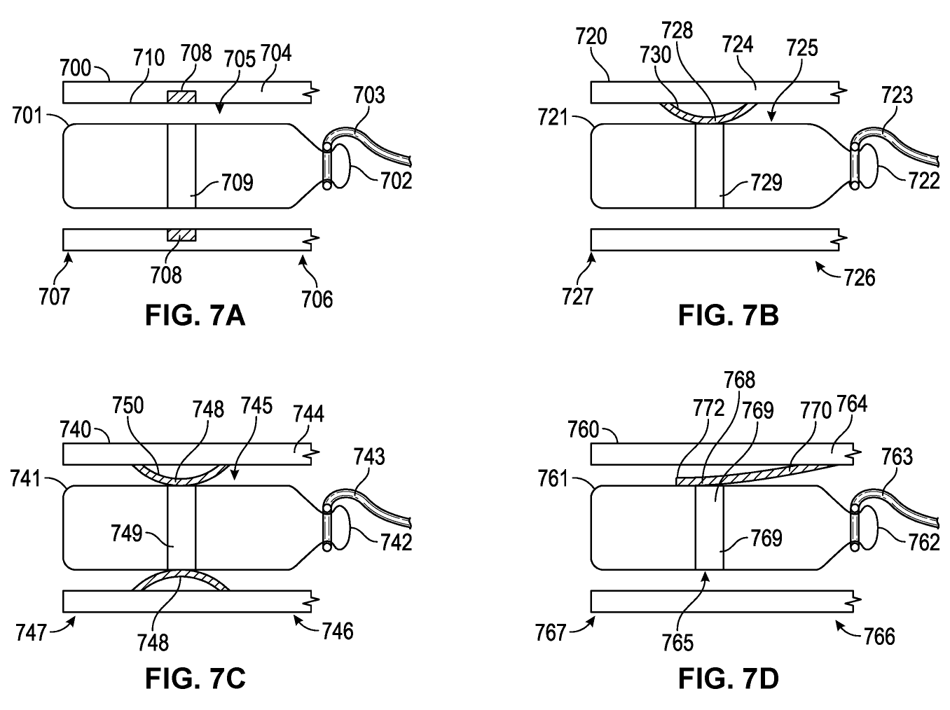

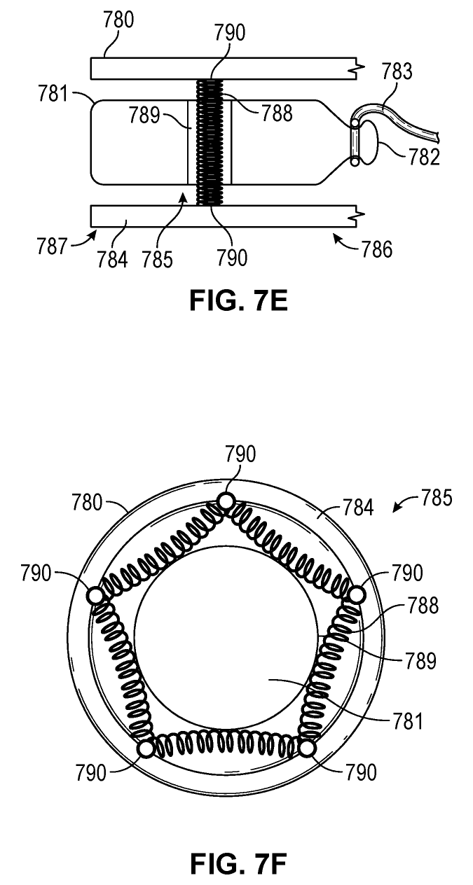

For example, in some embodiments, the hollow receptacle 528 and the medical device 502 are both cylindrical. The inner diameter of the hollow receptacle 528 can be slightly larger than the outer diameter of the medical device 502. In some embodiments, the medical device 502 has an outer diameter less than or equal to 15 mm, 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the outer diameter can be within a range from 1 mm to 15 mm, from 1 mm to 10 mm, or from 1 mm to 5 mm. In some embodiments, the outer diameter is less than or equal to 30 Fr (10 mm), 20 Fr (6.67 mm), 14 Fr (5 mm), 12 Fr (4 mm), 10 Fr (3.33 mm), 9 Fr (3 mm), 8 Fr (2.67 mm), 7 Fr (2.33 mm), 6 Fr (2 mm), 5 Fr (1.67 mm), 4 Fr (1.33 mm), 3 Fr (1 mm), or 2 Fr (0.67 mm). In some embodiments, the outer diameter of the medical device 502 is approximately 6.7 mm (about 20 Fr) and the hollow receptacle 528 has an inner diameter of approximately 7.7 mm (about 23 Fr), thus allowing approximately 0.5 mm of clearance between the outer surface of the medical device 502 and the inner surface of the hollow receptacle 528. The clearance between the outer surface of the medical device 502 and the inner surface of the hollow receptacle 528 allows for the medical device 502 to be received even if covered in material (e.g., overgrown tissue, clotted blood). Aspects of the hollow receptacle 528 discussed herein, including a cutting element (shown, for example, in FIG. 7D), can aid in removing material encapsulating the medical device 502, and thereby reduce the extent of clearance for the hollow receptacle 528 to receive the implanted medical device 502.

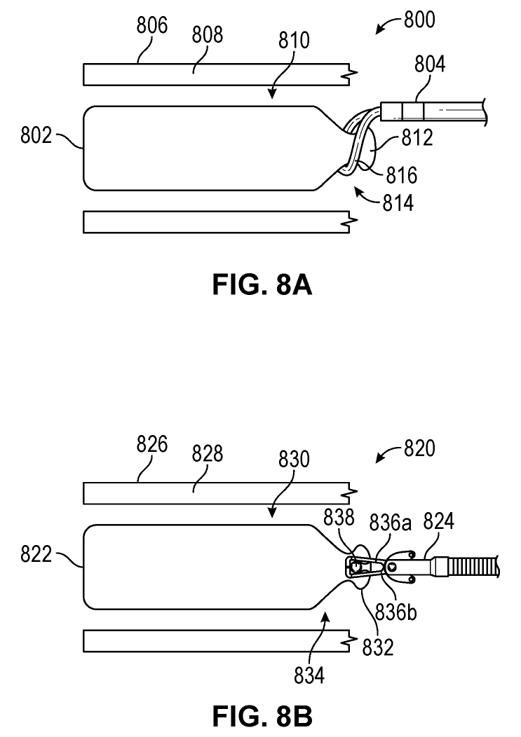

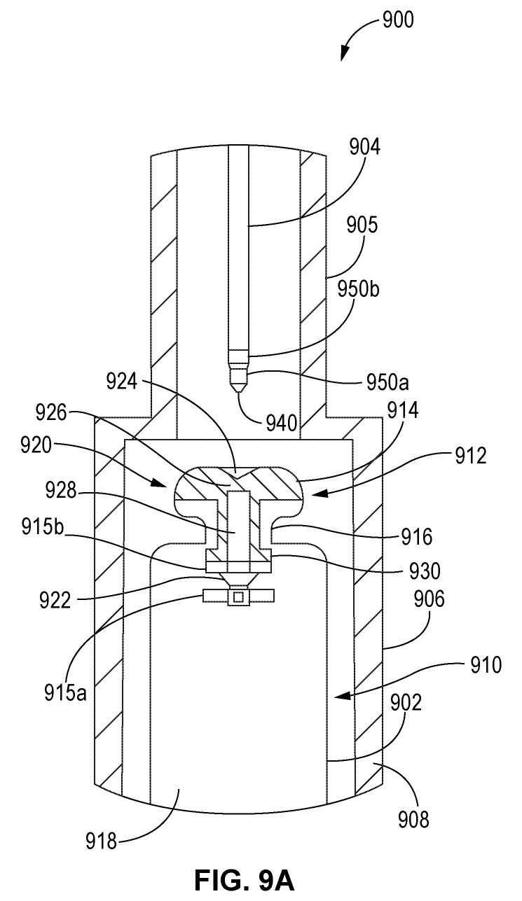

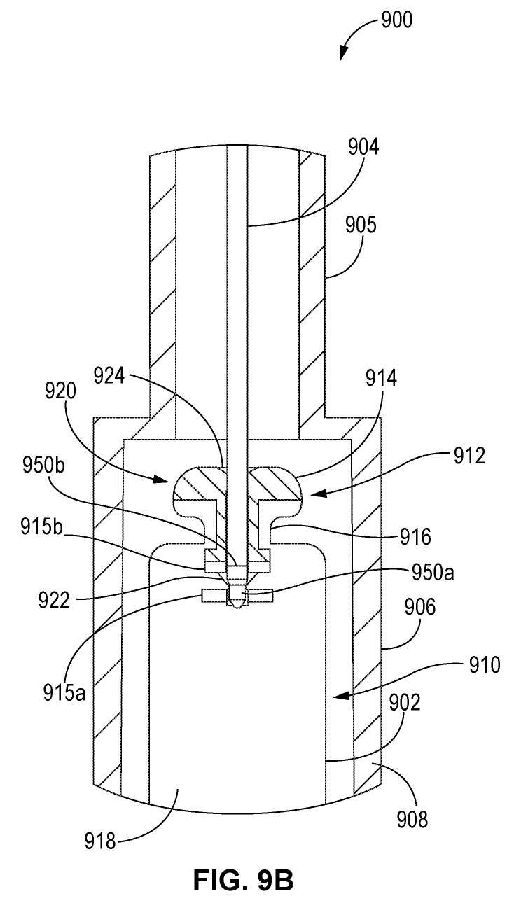

To aid in coupling to the medical device 502, the system 500 can further include a capture element 532 configured to releasably couple to a portion of the medical device 502 (e.g., to the attachment mechanism 510 at the proximal end 508 of the housing 506). The capture element 532 can be any component configured to mechanically couple to a portion of the medical device 502, such as a snare, a loop, pincers, etc. Additional details regarding example embodiments of the capture element 532 are described herein with reference to FIGS. 8A–9B.

The capture element 532 can be positioned within the lumen 530 of the elongate shaft 520. The capture element 532 can be connected to a tether 534 (e.g., a wire, push rod) within the lumen 530 of the elongate shaft 520 that extends from a proximal end of the system 500 (e.g., the proximal end 522 of the elongate shaft 520) to the capture element 532. The proximal end of the tether 534 can be connected to controls (not shown) that may be manipulated by an operator and/or robotically to control the capture element 532.

The capture element 532 can cooperate with the hollow receptacle 528 to couple to the medical device 502, e.g., the capture element 532 can be used to temporarily secure the medical device 502 so that the hollow receptacle 528 can be advanced over the medical device 502. For example, in some embodiments, the capture element 532 is introduced to the target site within the lumen 530 of the elongate shaft 520, and is then extended beyond the distal end 526 of the elongate shaft 520 and out of the hollow receptacle 528 to engage with (e.g., mechanically couple to) a portion of the medical device 502 (e.g., the attachment mechanism 510). Once the medical device 502 and capture element 532 are coupled, the hollow receptacle 528 can be guided distally over the capture element 532 to reach and receive the medical device 502. In some embodiments, when the medical device 502 is partially contained within the hollow receptacle 528, the capture element 532 can be used to draw the medical device 502 further into the hollow receptacle 528 (e.g., by pulling the medical device 502 proximally relative to the elongate shaft 520).

To deliver electrical energy to the medical device 502 while it is received within the hollow receptacle 528, the system 500 includes one or more electrodes (e.g., ring electrodes, patch electrodes, pin electrodes, ball electrodes) that can be electrically coupled to the medical device 502. As shown in the example embodiment of FIG. 5, the system 500 can include a first electrode 536a configured to electrically couple to the medical device 502 (e.g., via the first portion 516a of the medical device 502), and a second electrode 536b configured to electrically couple to the medical device 502 (e.g., via the second portion 516b of the medical device 502) (collectively, “electrodes 536”). The first electrode 536a may be located on the hollow receptacle 528, and the second electrode 536b may be located on the capture element 532. Accordingly, when the capture element 532 is coupled to the proximal end 508 of the housing 506, the second electrode 536b can be coupled to the second portion 516b of the housing 506. Subsequently, when the hollow receptacle 528 is guided over the capture element 532 to receive the medical device 502, the first electrode 536a can be aligned with and coupled to the first portion 516a of the housing 506.

In other embodiments, however, the configuration of the electrodes 536 may be varied as desired. For example, the first and second electrodes 536a, 536b can both be located on the hollow receptacle 528, such that the capture element 532 does not include any electrodes 536. Moreover, the system 500 can include a different number of electrodes 536 (e.g., one, three, four, five, or more electrodes 536). The number and locations of the electrodes 536 of the system 500 can be selected based on the configuration of the medical device 502 (e.g., based on the number of locations of the portions 516), so that when brought into alignment, electrical energy can be delivered from each of the electrodes 536 of the system 500 to the corresponding portions 516 of the medical device 502. Example configurations of electrodes of the hollow receptacle 528 and capture element 532 are described further herein with reference to FIGS. 7A–9B.

In some embodiments, the electrodes 536 are configured to deliver electrical energy to the medical device 502 via contact between the electrode 536 and the corresponding portion 516 of the medical device 502. As used herein, “contact” includes physical contact between the electrode 536 and the medical device 502, as well as electrical contact between the electrode 536 and the medical device 502 in situations where there may be a small spatial separation between the electrode 536 and medical device 502 (e.g., due to the presence of blood, encapsulating tissue) that does not substantially affect the transmission of electrical energy to the medical device 502.

The configuration of the electrodes 536 of the system 500 can optionally be selected to ensure patient safety during delivery of electrical energy. In some embodiments, electrical energy may only be delivered when both electrodes contact the respective portions 516 of the medical device 502. For example, in embodiments in which the first electrode 536a of the system 500 is on the hollow receptacle 528 and the second electrode 536b of the system 500 is on the capture element 532, no electrical energy may be delivered through the electrodes 536 unless both are electrically coupled respectively to the first and second portions 516a, 516b of the medical device 502. Such a two-part electrode arrangement can safeguard against accidental discharge of electrical energy (e.g., while introducing the elongate shaft 520 within the body of the patient, receiving a portion of the medical device 502 in the hollow receptacle 528).

Electrical energy from the external power source 524 can be delivered to the electrodes 536 by one or more electrical connectors, such as wires and/or conductive traces. For example, a first electrical connector 538a can be used to deliver electrical energy to the first electrode 536a, and a second electrical connector 538b can be used to deliver electrical energy to the second electrode 536b (collectively, “electrical connectors 538”). The first electrical connector 538a may be embedded within a wall of the elongate shaft 520, and the second electrical connector 538b may be incorporated into the tether 534 of the capture element 532. In other embodiments, the number and location of the electrical connectors 538 may be varied as desired, depending on the locations of the corresponding electrodes 536. In some embodiments, the one or more electrical connectors 538 are coupled to the external power source 524, extend distally along or within the elongate shaft 520, and terminate by coupling to the corresponding electrode 536 at a distal end of the system 500 (e.g., the first electrode 536a or the second electrode 536b).

Delivering electrical energy to the medical device 502 can charge the internal power source 504 of the medical device 502 while the medical device 502 remains implanted within the patient. Alternatively or in combination, the delivered electrical energy can be used to directly power the operation of the medical device 502 without charging the power source 504 (e.g., electrical energy can be provided directly to an internal component of the medical device 502 without going through the power source 504). Alternatively or in combination, the delivered electrical energy can be used for other purposes, such as communicating with the medical device 502 while the device 502 is implanted. For instance, the communication can include delivering electrical signals to the medical device 502 (e.g., to program the medical device 502) and/or receiving electrical signals from the medical device 502 (e.g., to perform diagnostics of the status of the medical device 502 and/or to receive data from the medical device 502).

An example method for providing power to the medical device 502 in the body of the patient is as follows. The method can include introducing the distal end 526 of the elongate shaft 520 within the body of the patient, receiving at least a portion of the medical device 502 within the hollow receptacle 528 (e.g., after coupling the capture element 532 to the medical device 502), coupling the first electrode 536a to the first portion 516a of the medical device 502, coupling the second electrode 536b to the second portion 516b of the medical device 502, and providing electrical energy (e.g., power) to the medical device 502. In some embodiments, the second electrode 536b is coupled to the second portion 516b of the medical device 502 before the first electrode 536a is coupled to the first portion 516a of the medical device 502 (e.g., if the second electrode 536b is on the capture element 532 and the first electrode 536a is on the hollow receptacle 528). In some embodiments, the first electrode 536a is coupled to the first portion 516a before the second electrode 536b is coupled to the second portion 516b, or the first electrode 536a is coupled to the first portion 516a concurrently with the second electrode 536b being coupled to the second portion 516b (e.g., if the first and second electrodes 536a, 536b are both on the hollow receptacle 528).

FIGS. 6A–9B illustrate various configurations for components that may be incorporated in a power delivery system and/or medical device, such as the system 500 and/or the medical device 502 of FIG. 5. Any of the embodiments of FIGS. 6A–9B may be combined with the embodiment of FIG. 5. Moreover, any of the embodiments of FIGS. 6A–9B may be combined with each other.

FIGS. 6A–6D are partially schematic illustrations of medical devices that are configured to couple to a power delivery system in accordance with embodiments of the present technology. For example, the medical devices shown in FIG. 6A–6D may be used as the medical device 502 of FIG. 5. As described herein, the medical devices may include one or more conductive portions that are configured to electrically couple to one or more corresponding electrodes of the power delivery system to establish electrical contact. Because of the sensitive placement of the medical device (e.g., implanted within the body), the one or more portions of the medical device can be arranged for safety considerations, e.g., to avoid shorting, to ensure that no current can stray dangerously away from the medical device while electrical energy is delivered, etc.

FIG. 6A shows a side view of an example medical device 600, in accordance with embodiments of the present technology. The medical device 600 includes a housing 601 with a proximal end 602, a distal end 603, and an intermediate region 604 therebetween. The housing 601 includes a first portion 605a and a second portion 605b that are each configured to receive electrical energy from a power delivery system (e.g., the system 500 of FIG. 5). An insulating region 606 electrically isolates the first and second portions 605a, 605b so that the portions 605a, 605b may be individually electrically addressable.

In the example shown in FIG. 6A, the first portion 605a is located at the intermediate region 604 of the housing 601. The first portion 605a can have an annular shape that extends fully around a perimeter (e.g., circumference) of the device 600. The second portion 605b can be located at the distal end 602 of the housing 601 (e.g., on an attachment mechanism 607 that is configured to couple to a capture element of the power delivery system). The insulating region 606 can be an annular portion of the housing 601 between the first portion 605a and the second portion 605b.

The configuration in FIG. 6A may be used in embodiments where the power delivery system includes a first electrode on a hollow receptacle for contacting the first portion 605a, and a second electrode on a capture element for contacting the second portion 605b (e.g., identical or similar to the configuration shown in FIG. 5). The annular shape of the first portion 605a may be advantageous to ensure good electrical contact with the first electrode even when the hollow receptacle is rotated to different orientations relative to the medical device 600, thereby providing more flexible and convenient delivery of electrical energy to the medical device 600.

FIG. 6B shows a side view of another example of a medical device 610, in accordance with embodiments of the present technology. The medical device 610 includes a housing 611 with a proximal end 612, a distal end 613, and an intermediate region 614 therebetween, which may be identical or generally similar to the corresponding components of FIG. 6A. The housing 611 includes a first portion 615a and a second portion 615b that are each configured to transmit and receive electrical energy. An insulating region 616 electrically isolates the first and second portions 615a, 615b so that the portions 615a, 615b may be individually electrically addressable.

In the example shown in FIG. 6B, both the first and second portions 615a, 615b are located at the intermediate region 614 of the housing 611. The first and second portions 615a, 615b can be spaced apart from each other by a distance along a longitudinal axis of the device 610, e.g., the first portion 615a may be distal to the second portion 615b as shown in FIG. 6B. The first and second portions 615a, 615b can each have an annular shape that extends fully around a perimeter (e.g., circumference) of the medical device 610. The insulating region 616 can be the annular portion of the housing 611 between the first portion 615a and the second portion 615b.

The configuration shown in FIG. 6B may be used in embodiments where the power delivery system includes two electrodes on a hollow receptacle for contacting the first and second portions 615a and 615b. The annular shape of the first and second portions 615a, 615b may be advantageous to ensure good electrical contact with the electrodes even when the hollow receptacle is rotated to different orientations relative to the medical device 600, thereby providing more flexible and convenient delivery of electrical energy to the medical device 600. In such embodiments, the capture element of the power delivery system may not include any electrodes, such that the capture element is used solely for mechanical coupling purposes.

FIG. 6C shows a side view of a further example of a medical device 620, in accordance with embodiments of the present technology. The medical device 620 includes a housing 621 with a proximal end 622, a distal end 623, and an intermediate region 624 therebetween, which may be identical or generally similar to the corresponding components of FIG. 6A. The housing 621 includes a first portion 625a and a second portion 625b that are each configured to transmit and receive electrical energy. An insulating region 626 electrically isolates the first and second portions 625a, 625b so that the portions 625a, 625b can be individually electrically addressable.

In the example shown in FIG. 6C, the first portion 625a is located at the intermediate region 624 of the housing 621. In some embodiments, the first portion 625a takes the form of a single discrete area on the housing 621 (e.g., an electrically conductive patch). The single discrete area of the first portion 625a can have any suitable shape (e.g., a circle, an oval, a rectangle, a square, a triangle, a partial annulus). The second portion 625b can be located at the proximal end 622 of the housing 621 (e.g., on an attachment mechanism 627 that is configured to couple to a capture element of the power delivery system). The insulating region 626 can be the remaining portion of the housing 621 between the first portion 625a and the second portion 625b.

The configuration in FIG. 6C may be used in embodiments where the power delivery system includes a first electrode on a hollow receptacle for contacting the first portion 625a, and a second electrode on a capture element for contacting the second portion 625b (e.g., identical or similar to the configuration shown in FIG. 5). In such embodiments, the first electrode of the hollow receptacle may have an annular shape to ensure good electrical contact with the first portion 625a even when the hollow receptacle is rotated to different orientations relative to the medical device 620. Alternatively, the first electrode of the hollow receptacle may be a patch electrode (or other electrode type) having a shape similar to the shape of the first portion 625a.

FIG. 6D shows a side view of yet another example of a medical device 630, in accordance with embodiments of the present technology. The medical device 630 includes a housing 631 with a proximal end 632, a distal end 633, and an intermediate region 634 therebetween, which may be identical or generally similar to the corresponding components of FIG. 6A. The housing 631 includes a first portion 635a and a second portion 635b that are each configured to transmit and receive electrical energy. An insulating region 636a electrically isolates the first and second portions 635a, 635b so that the portions 635a, 653b can be individually electrically addressable.

In the example shown in FIG. 6D, the first portion 635a is located at the intermediate region 634 of the housing 631, and the second portion 635b is located at the proximal end 632 of the housing 631 (e.g., on an attachment mechanism 637 that is configured to couple to a capture element of the power delivery system). In some embodiments, the first portion 635a takes the form of a plurality of discrete areas on the housing 631 (e.g., a plurality of electrically conductive patches). The discrete areas can each have any suitable shape (e.g., a circle, an oval, a rectangle, a square, a triangle, a partial annulus). Each of the individual discrete areas of the first portion 635a can be electrically isolated from other individual discrete areas by an insulating region 636b therebetween, so that each of the individual discrete areas of the plurality of discrete areas can be individually electrically addressable. The insulating region 636b can be contiguous with the insulating region 636a, and can be made of the same or different materials as the insulating region 636a.

Although FIG. 6D illustrates two discrete areas, the first portion 635a can be composed of any suitable number of discrete areas, such as three, four, five, or more discrete areas. Moreover, although FIG. 6D illustrates the discrete areas as being at the same longitudinal position and different circumferential positions on the housing 631, other configurations may be used, e.g., the discrete areas may be at the same circumferential position and at different longitudinal positions, the discrete areas may be at different longitudinal and circumferential positions, etc. The discrete areas may be arranged in any suitable pattern, such as a ring, band, line, spiral, array, grid, etc.

The configuration in FIG. 6D may be used in embodiments where the power delivery system includes a first electrode on a hollow receptacle for contacting the first portion 635a, and a second electrode on a capture element for contacting the second portion 635b. In such embodiments, the first electrode of the hollow receptacle may optionally have an annular shape to ensure good electrical contact with most or all of the discrete areas of the first portion 635a even when the hollow receptacle is rotated to different orientations relative to the medical device 630. Alternatively, depending on the number, shape, and configuration of the discrete areas, other shapes for the first electrode may be used, as long as the shape is sufficiently large to contact some or all of the discrete areas concurrently. In other embodiments, the hollow receptacle may include a plurality of first electrodes that are each configured to contact a respective discrete area of the first portion 35a.

Although FIGS. 6A–6D illustrate medical devices with two conductive portions for contacting two electrodes of a power delivery system, the medical devices herein may include any suitable number of conductive portions, such as three, four, five, or more conductive portions. Each conductive portion may have any suitable size and shape, e.g., some or all of the conductive portions may have the same size and shape, or some or all of the conductive portions may have different sizes and/or shapes. Each conductive portion may be located at any suitable location of the medical device. The number, geometry, and arrangement of the conductive portions may be selected depending on the corresponding arrangement of electrodes of the power delivery system, to avoid interfering with the functions of the medical device (e.g., in embodiments where the medical device itself includes electrodes for delivering electrical energy to tissue, it may be desirable to locate the conductive portions away from such electrodes), and/or any other relevant design considerations.

FIGS. 7A–7F are partially schematic illustrations of hollow receptacles of a power delivery system in accordance with embodiments of the present technology. For example, the hollow receptacles shown in FIGS. 7A–7F may be used as the hollow receptacle 528 of the system 500 of FIG. 5. As described herein, the hollow receptacles can include one or more electrodes to deliver electrical energy to a medical device that is received within the hollow receptacle. The electrodes can be arranged so that the electrodes align with one or more corresponding conductive portions of the medical device when the medical device is positioned within the hollow receptacle.

FIG. 7A shows a side cross-sectional view of an example hollow receptacle 700 of a power delivery system in accordance with embodiments of the present technology. The hollow receptacle 700 can be configured to receive a medical device 701. The medical device 701 can include an attachment mechanism 702 (e.g., at a distal end of the medical device 701) for coupling to a capture element 703 (e.g., a snare) that facilitates capture and retention of the medical device 701 within the hollow receptacle 700. The hollow receptacle 700 includes a wall 704 configured to surround at least a portion of the medical device 701 and a lumen 705 configured to receive at least a portion of the medical device 701. The wall 704 and the lumen 705 can extend from a proximal end 706 of the hollow receptacle 700 to a distal end 707 of the hollow receptacle 700.

The hollow receptacle 700 includes an electrode 708 that is configured to electrically couple to a portion 709 of the medical device 701 (the portion 709 is depicted as being annular merely for illustrative purposes; in other embodiments, the portion 709 can have a different shape, such as any of the shapes discussed with respect to FIGS. 6A–6D). In the embodiment of FIG. 7A, the electrode 708 is a ring electrode that extends partially or fully around the perimeter (e.g., circumference) of the lumen 705. This configuration may be advantageous, for example, to ensure good electrical contact with the portion 709 even when the hollow receptacle 700 is rotated to different orientations relative to the medical device 701. In other embodiments, however, the electrode 708 can be a different type of electrode, such an electrode that has a smaller discrete shape (e.g., a patch electrode).

As shown in FIG. 7A, the electrode 708 can be part of or embedded in the wall 704 of the hollow receptacle 700, such that a lumen-facing surface of the electrode 708 is substantially flush with an inner surface 710 of the hollow receptacle 700. This low-profile configuration can reduce the space within the lumen 705 occupied by the electrode 708, which may be advantageous for reducing or minimizing the overall dimensions of the hollow receptacle 700, e.g., to allow the hollow receptacle 700 to be navigated into small and/or narrow areas (e.g., lumens within the vasculature of a human patient) to reach a target location. In such embodiments, the diameter of the lumen 705 may be substantially equal to the outer diameter of the medical device 701 to ensure good contact between the electrode 708 and the portion 709 of the medical device 701 when the medical device 701 is received within the hollow receptacle 700.

FIG. 7B shows a side cross-sectional view of another example of a hollow receptacle 720 of a power delivery system in accordance with embodiments of the present technology. The hollow receptacle 720 can be configured to receive a medical device 721. The medical device 721 can include an attachment mechanism 722 (e.g., at a distal end of the medical device 721) for coupling to a capture element 723 (e.g., a snare) that facilitates capture and retention the medical device 721 within the hollow receptacle 720. The hollow receptacle 720 includes a wall 724 configured to surround at least a portion of the medical device 721 and a lumen 725 configured to receive at least a portion of the medical device 721. The wall 724 and the lumen 725 can extend from a proximal end 726 of the hollow receptacle 720 to a distal end 727 of the hollow receptacle 720.

The hollow receptacle 720 includes an electrode 728 that is configured to electrically couple to a portion 729 of the medical device 721 (the portion 729 is depicted as being annular merely for illustrative purposes; in other embodiments, the portion 729 can have a different shape, such as any of the shapes discussed with respect to FIGS. 6A–6D). In the embodiment of FIG. 7B, the electrode 728 is part of or is coupled to a spring element 730 that is coupled to an internal surface of the wall 724 of the hollow receptacle 720. The spring element 730 can be a wire, strip, sheet, or other component having elastic properties to bias the electrode 728 into contact with the portion 729 of the medical device 721. For example, the spring element 730 can be made of an elastic metal, an elastic polymer, or other suitable material.

In some embodiments, the spring element 730 is coupled to the wall 724 in a manner such that at least a portion of the spring element 730 protrudes inward toward the center of the lumen 725 of the hollow receptacle 720. Although FIG. 7B depicts spring element 730 as being coupled to the wall 724 at both ends to form a convex contact surface, the spring element 730 can alternatively be coupled to the wall 724 at a single end to form a cantilever. When the medical device 721 is positioned within the lumen 725, the medical device 721 can contact and deflect the spring element 730 outward away from the center of the lumen 725. The resistance of the spring element 730 to the deflection can produce a spring force that biases the electrode 728 against the portion 729 of the medical device 721, thereby maintaining good electrical and/or physical contact between these components. In such embodiments, the diameter of the lumen 725 may be larger than the outer diameter of the medical device 721 (e.g., to make it easier to fit the medical device 721 into the hollow receptacle 720) since the spring element 730 protrudes inward to ensure good contact between the electrode 728 and the portion 729 of the medical device 721 when the medical device 721 is received within the hollow receptacle 720.

FIG. 7C shows a side cross-sectional view of a further example of hollow receptacle 740 of a power delivery system in accordance with embodiments of the present technology. The hollow receptacle 740 is configured to receive a medical device 741. The medical device 741 can include an attachment mechanism 742 (e.g., at a distal end of the medical device 741) for coupling to a capture element 743 (e.g., a snare) that facilitates capture and retention of the medical device 741 within the hollow receptacle 740. The hollow receptacle 740 includes a wall 744 configured to surround at least a portion of the medical device 741 and a lumen 745 configured to receive at least a portion of the medical device 741. The wall 744 and the lumen 745 can extend from a proximal end 746 of the hollow receptacle 740 to a distal end 747 of the hollow receptacle 740.

The hollow receptacle 740 includes an electrode 748 that is configured to electrically couple to a portion 749 of the medical device 741 (the portion 749 is depicted as being annular merely for illustrative purposes; in other embodiments, the portion 749 can have a different shape, such as any of the shapes discussed with respect to FIGS. 6A–6D). The electrode 748 is part of or is coupled to a spring element 750 that is coupled to an internal surface of the wall 744 of the hollow receptacle 740. The electrode 748 and spring element 750 can be generally similar to the corresponding components of FIG. 7B, except that the electrode 748 and spring element 750 each have an annular shape that extends partially or fully around the perimeter (e.g., circumference) of the lumen 745. This configuration may be advantageous, for example, to ensure good electrical contact with the portion 749 even when the hollow receptacle 740 is rotated to different orientations relative to the medical device 741.

FIG. 7D shows a side cross-sectional view of yet another example of a hollow receptacle 760 of a power delivery system in accordance with embodiments of the present technology. The hollow receptacle 760 is configured to receive a medical device 761. The medical device 761 can include an attachment mechanism 762 (e.g., at a distal end of the medical device 761) for coupling to a capture element 763 (e.g., a snare) that facilitates capture and retention of the medical device 761 within the hollow receptacle 740. The hollow receptacle 760 includes a wall 764 configured to surround at least a portion of the medical device 761 and a lumen 765 configured to receive at least a portion of the medical device 761. The wall 764 and the lumen 765 can extend from a proximal end 766 of the hollow receptacle 760 to a distal end 767 of the hollow receptacle 760.

The hollow receptacle 760 includes an electrode 768 that is configured to electrically couple to a portion 769 of the medical device 761 (the portion 769 is depicted as being annular merely for illustrative purposes; in other embodiments, the portion 769 can have a different shape, such as any of the shapes discussed with respect to FIGS. 6A–6D). In the embodiment of FIG. 7D, the electrode 768 is part of or is coupled to a cantilever element 770 that is coupled to an internal surface of the wall 764 of the hollow receptacle 760. The cantilever element 770 can be a wire, strip, beam, or other component having a proximal end that is connected to the wall 764, and a free distal end that is not connected to the wall 764. The distal end can extend inwardly toward the center of the lumen 765.

In some embodiments, the cantilever element 770 is a spring element having elastic properties to bias the electrode 768 into contact with the portion 769 of the medical device 761, e.g., similar to the spring element 730 of FIG. 7B. In other embodiment, however, the cantilever element 770 can instead be a rigid component with little or no elasticity.

In some embodiments, the cantilever element 770 is configured as a cutting element to remove material on the medical device 761 that may impede electrical coupling of the electrode 768 to the medical device 761. In some cases, after implantation, the medical device 761 may become at least partially encapsulated in tissue, e.g., due to fibrotic overgrowth at the implantation site over time. The tissue can grow over the medical device 761 and make it difficult to establish physical and/or electrical contact between the medical device 761 and the electrode 768. To address this issue, the cantilever element 770 can include a sharp edge 772 at the free distal end that is configured to cut through tissue (or other material obstruction) covering the medical device 761. The sharp edge 772 of the cantilever element 770 may extend toward the center of the lumen 765 of the hollow receptacle 760. Accordingly, when the hollow receptacle 760 is advanced distally over the medical device 761, the sharp edge 772 can contact the external surface of the medical device 761 thus cutting through any material (e.g., tissue overgrowth) on the surface of the medical device 761.

Alternatively or in combination, other types of mechanisms may be used to clear tissue and/or other obstructions from the medical device 761, such as thermal ablation, radiofrequency ablation, cryoablation, etc. Such mechanisms may be incorporated into the hollow receptacle 760, or may be part of a separate component of the power delivery system.

FIGS. 7E and 7F show a side cross-sectional view and an end view, respectively, of a hollow receptacle 780 of a power delivery system in accordance with embodiments of the present technology. The hollow receptacle 780 is configured to receive a medical device 781. The medical device 781 can include an attachment mechanism 782 (e.g., at a distal end of the medical device 781) for coupling to a capture element 783 (e.g., a snare) that facilitates the medical device 781 within the hollow receptacle 780. The hollow receptacle 780 includes a wall 784 configured to surround at least a portion of the medical device 781 and a lumen 785 configured to receive at least a portion of the medical device 781. The wall 784 and the lumen 785 can extend from a proximal end 786 of the hollow receptacle 780 to a distal end 787 of the hollow receptacle 780.

The hollow receptacle 780 includes an electrode 788 that is configured to electrically couple to a portion 789 of the medical device 781 (the portion 789 is depicted as being annular merely for illustrative purposes; in other embodiments, the portion 789 can have a different shape, such as any of the shapes discussed with respect to FIGS. 6A–6D). In the illustrated embodiment, the electrode 788 is configured as a spring element (e.g., a helical spring) that is coupled to an internal surface of the wall 784 of the hollow receptacle 780. As best seen in FIG. 7F, the spring element can extend along the entire perimeter (e.g., circumference) of the lumen 785, thereby surrounding the medical device 781. The spring element may be connected to the internal surface of the wall 784 via one or more attachments 790. The spring element can have elastic properties to bias the electrode 788 into contact with the portion 789 of the medical device 781 (e.g., similar to the spring element 730 of FIG. 7B and/or the spring element 750 of FIG. 7C).

Although FIGS. 7A–7F illustrate hollow receptacles with a single electrode for contacting a corresponding conductive portion of a medical device, the hollow receptacles herein may include any suitable number of electrodes, such as three, four, five, or more electrodes. Each electrode may have substantially the same size and shape as the corresponding conductive portion, or the electrode may be larger or smaller than the corresponding conductive portion. Each electrode may have any configuration, e.g., some or all of the electrodes may have the same shape or different shapes, any of the electrodes may be mounted on spring elements and/or cutting elements, etc. Each electrode may be located at any suitable location of hollow receptacle. The number, geometry, and arrangement of the electrodes may be selected depending on the corresponding arrangement of conductive portions of the medical device, e.g., the electrodes can be at locations that overlap or are otherwise aligned with the locations of the conductive portions when the medical device is received within the hollow receptacle.

FIGS. 8A and 8B are partially schematic illustrations of capture elements of a power delivery system in accordance with embodiments of the present technology. For example, the capture elements shown in FIGS. 8A and 8B may be used as the capture element 532 of the system 500 of FIG. 5. As described herein, the capture elements can be configured to capture a medical device by coupling to at least a portion of the medical device (e.g., an attachment mechanism). Optionally, the capture element may include one or more electrodes configured to couple to one or more corresponding conductive portions of the medical device to transfer electrical energy thereto when the medical device is coupled to the capture element.

FIG. 8A shows a side cross-sectional view of an example power delivery system 800 configured to receive a medical device 802 in accordance with embodiments of the present technology. The system 800 can couple (e.g., mechanically and/or electrically couple) to the medical device 802 via a capture element 804. In some embodiments, the system 800 includes an elongate shaft (not shown) with a hollow receptacle 806 having a wall 808 and a lumen 810 sized and shaped to receive at least a portion of the medical device 802. The hollow receptacle 806 may be advanced over the medical device 802 once the medical device 802 is captured by the capture element 804.

The capture element 804 can be configured to couple to a portion of the medical device 802, such as an attachment mechanism 812 (e.g., a protrusion, flange, opening) at a proximal end 814 of the medical device 802. In the example illustrated in FIG. 8A, the attachment mechanism 812 is configured as a flange with a narrowed neck, and the capture element 804 includes a snare 816 configured to loop around the neck of the flange. The snare 816 can be tightened around the flange to form a secure, temporary mechanical coupling to the medical device 802. Optionally, the snare 816 can include an electrode, such that the snare 816 can be used to deliver electrical energy to the medical device 802 (e.g., to a conductive portion of the attachment mechanism 812). In such embodiments, the snare 816 may be made partially or entirely out of an electrically conductive material (e.g., metal or a conductive polymer), and may be electrically coupled to an external power source (not shown). In other embodiments, however, the snare 816 may not include any electrodes and thus may serve solely for mechanical coupling to the medical device 802.

FIG. 8B shows a side cross-sectional view of an example power delivery system 820 configured to receive a medical device 822 in accordance with embodiments of the present technology. The system 820 can couple (e.g., mechanically and/or electrically couple) to the medical device 822 via a capture element 824. In some embodiments, the system 820 includes an elongate shaft (not shown) with a hollow receptacle 826 having a wall 828 and a lumen 830 sized and shaped to receive at least a portion of the medical device 822. The hollow receptacle 826 may be advanced over the medical device 822 once the medical device 822 is captured by the capture element 824.

The capture element 824 can be configured to couple to a portion of the medical device 822, such as an attachment mechanism 832 (e.g., a protrusion, flange, opening) at a proximal end 834 of the medical device 822. In the example illustrated in FIG. 8B, the capture element 824 is configured as a set of pincers including a first jaw 836a and a second jaw 836b. The jaws 836a, 836b can be configured to clamp around a portion of the attachment mechanism 832, such as to a pin 838 on the attachment mechanism 832, to form a secure, temporary mechanical coupling to the medical device 822. Optionally, one or both jaws 836a, 836b can serve as an electrode, such that the jaws 836a, 836b can be used to deliver electrical energy to the medical device 822 (e.g., via the pin 838, which may be made of a conductive material). In such embodiments, the jaws 836a, 836b may be made partially or entirely out of an electrically conductive material (e.g., metal or a conductive polymer), and may be electrically coupled to an external power source (not shown). In other embodiments, however, the jaws 836a, 836b may not serve as electrodes and thus may serve solely for mechanical coupling to the medical device 822.

FIGS. 9A and 9B are partially schematic illustrations of capture elements of a power delivery system in accordance with embodiments of the present technology. For example, the capture elements shown in FIGS. 9A and 9B may be used as the capture element 532 of the system 500 of FIG. 5, and/or as an electrode delivery shaft or charging shaft of the capture element 532, e.g. in place of the electrical connector 538b, as further described herein. The capture elements shown in FIGS. 9A and 9B can, in some embodiments, be similar in structure, function and use to the capture element 532 of the system 500 of FIG. 5, while incorporating the herein-described features and modifications. As described herein, the capture elements can be configured to capture a medical device by coupling to at least a portion of the medical device (e.g., an attachment mechanism). Optionally, the capture element may include one or more electrodes configured to couple to one or more corresponding conductive portions of the medical device to transfer electrical energy thereto when the medical device is coupled to the capture element.

FIGS. 9A and 9B show side cross-sectional views of an example power delivery system 900 configured to receive a medical device 902 (e.g., leadless pacemaker) in accordance with embodiments of the present technology. The system 900 can couple (e.g., mechanically and/or electrically couple) to the medical device 902 via a capture element 904. The system 900 includes an elongate shaft 905 with a hollow receptacle 906 having a wall 908 and a lumen 910 sized and shaped to receive at least a portion of the medical device 902. The hollow receptacle 906 may be advanced over the medical device 902 once the medical device 902 is captured by the capture element 904 (and/or the capture element 904 may be advanced to capture the medical device 902 once the hollow receptacle 906 has been advanced over the medical device 902).

The medical device 902 can be similar in structure, function and use to any of the other medical devices described or depicted herein, e.g. the pacing devices 200, 300, 400 or medical device 502, with the additional and/or alternative features described herein pertaining to the medical device 902. The medical device 902 can include an attachment mechanism or attachment member 912 having a head or flange 914 at the proximal end, which is coupled via a neck 916 to the proximal end of the medical device housing 918. The diameter of the neck 916 can be smaller than the diameter of the head 914, and the diameter of the head 914 can be smaller than the diameter of the housing 918.

A polymeric seal 920 can form at least a portion of the proximal end of the head 914, and extend distally through the neck 916 to a socket or receptacle 922 where one or both of a first portion 915a and a second portion 915b is/are disposed. The first portion 915a and second portion 915b is/are configured as electrical contact(s) which transmit and receive electrical energy. The first and second portions 915a, 915b are electrically isolated from each other so as to be individually electrically addressable.

The polymeric seal 920 may form a tapered recess 924 on its proximal end surface, a relatively thin pierceable septum 926 distal of and adjacent to the recess 924, and a hollow passage 928 distal of and adjacent to the septum 926. The passage 928 provides access to the socket 922 which is disposed at the distal end of the passage 928. The polymeric seal 920 may form a distal flange 930 which cooperates with an overlapping portion of the neck 916 or housing 918 to retain the polymeric seal in position on the medical device 902 against longitudinal forces.

As an alternative to the depicted polymeric seal 920 with the hollow passage 928 and pierceable septum 926, the polymeric seal 920 can be configured with a self-sealing passage or septum which is liquid-tight and formed by sidewalls which are in close contact with each other (e.g., similar to a hemostatic valve). The sidewalls can spread apart when the capture element 904 is inserted into the polymeric seal, forming a liquid-light seal against the capture element 904 while it is present, and then re-form a liquid-tight seal when the capture element 904 has been removed, via close adherence of the sidewalls to each other.

The capture element 904 can be configured to couple electrically and/or mechanically to the medical device 902, via insertion through the polymeric seal 920 to the portion(s) 915a and/or 915b. The capture element 904 can include a conductive wire or other shaft with a pointed distal tip 940, and a first electrode 950a located at or near the distal tip 940, e.g. at a narrowed-diameter distal section of the capture element 904. A second electrode 950b can be located proximal of the first electrode 950a, e.g., at the distal end of a larger-diameter proximal portion of the capture element 904. When multiple electrodes 950 are employed, the capture element 904 can be configured as a coaxial shaft with corresponding multiple conduction paths to the respective electrodes, which paths and electrodes are electrically isolated from each other. When a single electrode (e.g., the first electrode 950a) is employed on the capture element 904, a second electrode may be disposed on or in the hollow receptacle 906, to couple electrically with a portion on the exterior of the medical device housing 918, e.g. as shown in FIGS. 7A–7F.

In some embodiments, the depicted capture element 904 can be implemented as a component of a capture element such as the capture element 532 of FIG. 5. For example, the various described embodiments of the capture element 904 can be implemented in place of the electrical connector 538b. The capture element 904 can be disposed, e.g., slidably disposed, as an electrode-tipped shaft or wire extending within a lumen passing through the capture element 532, e.g., through the tether 534, and be employed to deliver electrical energy to the medical device 902 as described herein. In such embodiments, the balance of the components of the capture element 532 can be configured and function as described herein. Alternatively, the capture element 904 can be employed alongside, but not passing through the capture element 532, and function to deliver electrical energy to the medical device 902.

To deliver electrical energy to the medical device 902, the capture element 904 can be inserted into the proximal end of the medical device 902, through the polymeric seal 920. The pointed distal tip 940 can be placed in the recess 924 and pushed distally to pierce the septum 926 and advance through the passage 928 until the distal end portion of the capture element 904 is in the socket 922. Where the polymeric seal is configured with a hemostatic, self-sealing passage, the distal tip of the capture element 904 can push the sidewalls of the passage apart without tearing or puncturing the polymeric seal, and similarly advance to the socket 922. Once the distal end portion of the capture element 904 is in the socket 922 (see FIG. 9B), the first electrode 950a contacts and electrically couples with the first portion 915a and, where the respective components are present, the second electrode 950b contacts and electrically couples with the second portion 915b. Where the power delivery system 900 is so configured, a second electrode disposed on or in the hollow receptacle 906 to couples electrically with a portion on the exterior of the medical device housing 918. At this point, electrical current can be passed from an external power source through the capture element 904, through the electrical connection made via the electrode(s) 950a, 950b and the portion(s) 915a, 915b, to the battery in the medical device 902. In this manner, the battery can be charged or recharged as desired.

Although certain embodiments of the present technology are described in terms of a power delivery system including at least one electrode on a hollow receptacle, this is not intended to be limiting. In some embodiments, the systems herein may include two or more electrodes on a capture element, such that the hollow receptacle need not include any receptacles and may even be omitted altogether. Optionally, the system may include two capture elements, each with a respective electrode (e.g., one electrode on the capture element 804 of FIG. 8A and one electrode on the capture element 824 of FIG. 8B).

### Examples

The following examples are included to further describe some aspects of the present technology, and should not be used to limit the scope of the technology.

Example 1. A system for providing power to a medical device in a body of a patient, the system comprising:
an elongate shaft comprising a distal end that is configured to be positioned proximate to the medical device in the body of the patient;
a hollow receptacle at the distal end of the elongate shaft, wherein the hollow receptacle is configured to receive at least a portion of the medical device;
a first electrode configured to couple to a first portion of the medical device, wherein the first electrode is located on the hollow receptacle;
a second electrode configured to couple to a second portion of the medical device; and
an external power source configured to be electrically connected to the first and second electrodes to provide power to the medical device.

Example 2. The system of Example 1, wherein the first electrode is positioned at a first location on the hollow receptacle, and wherein the second electrode is positioned at a second location on the hollow receptacle different from the first location.

Example 3. The system of Example 2, wherein the first location and second locations are spaced apart from each other along a longitudinal axis of the hollow receptacle.

Example 4. The system of Example 1, further comprising a capture element configured to releasably couple to the second portion of the medical device, wherein the second electrode is located on the capture element.

Example 5. The system of Example 4, wherein the second portion is a proximal end of the medical device.

Example 6. The system of Example 4 or 5, wherein the elongate shaft comprises a lumen, and wherein the capture element is positioned at least partially within the lumen.

Example 7. The system of any one of Examples 1 to 6, wherein the first electrode is configured to directly contact the first portion of the medical device, and wherein the second electrode is configured to directly contact the second portion of the medical device.

Example 8. The system of any one of Examples 1 to 7, wherein the hollow receptacle is configured to extend around a circumference or perimeter of the medical device.

Example 9. The system of Example 8, wherein the first electrode comprises a ring electrode that extends around the circumference or the perimeter of the medical device.

Example 10. The system of any one of Examples 1 to 9, further comprising a spring element, wherein the spring element is configured to bias one or more of the first electrode or the second electrode into contact with the first portion or the second portion, respectively, of the medical device.

Example 11. The system of any one of Examples 1 to 10, further comprising a cutting element configured to remove tissue overgrowth on the medical device.

Example 12. The system of any one of Examples 1 to 11, wherein the first and second portions are part of a housing of the medical device.

Example 13. The system of Example 12, wherein the housing comprises an insulating region configured to electrically isolate the first and second portions from each other.

Example 14. The system of any one of Examples 1 to 13, wherein the medical device comprises an internal power source, and wherein the power provided by the external power source is configured to charge the internal power source.

Example 15. The system of any one of Examples 1 to 14, wherein the medical device is a leadless pacemaker.

Example 16. A method for providing power to a medical device in a body of a patient, the method comprising:
introducing a distal end of an elongate shaft within the body of the patient;
receiving at least a portion of the medical device within the body of the patient within a hollow receptacle at the distal end of the elongate shaft;
coupling a first electrode to a first portion of the medical device, wherein the first electrode is located on the hollow receptacle;
coupling a second electrode to a second portion of the medical device; and
providing power to the medical device via the first and second electrodes.

Example 17. The method of Example 16, wherein the first electrode is positioned at a first location on the hollow receptacle, and wherein the second electrode is positioned at a second location on the hollow receptacle different from the first location.

Example 18. The method of Example 17, wherein the first location and second locations are spaced apart from each other along a longitudinal axis of the hollow receptacle.

Example 19. The method of Example 16, further comprising coupling to the second portion of the medical device using a capture element, wherein the second electrode is located on the capture element.

Example 20. The method of Example 19, wherein the second portion is a proximal end of the medical device.

Example 21. The method of Example 19 or 20, wherein the capture element is positioned at least partially within a lumen of the elongate shaft.

Example 22. The method of any one of Examples 16 to 21, wherein the first electrode is configured to directly contact the first portion of the medical device, and wherein the second electrode is configured to directly contact the second portion of the medical device.

Example 23. The method of any one of Examples 16 to 22, wherein the hollow receptacle is configured to extend around a circumference or perimeter of the medical device.

Example 24. The method of Example 23, wherein the first electrode comprises a ring electrode that extends around the circumference or the perimeter of the medical device.

Example 25. The method of any one of Examples 16 to 24, further comprising biasing one or more of the first electrode or the second electrode into contact with the first portion or second portion, respectively, of the medical device.

Example 26. The method of any one of Examples 16 to 25, further comprising removing tissue overgrowth on the medical device before coupling one or more of the first electrode or the second electrode to the medical device.

Example 27. The method of any one of Examples 16 to 26, wherein the first and second portions are part of a housing of the medical device.

Example 28. The method of Example 27, wherein the housing comprises an insulating region configured to electrically isolate the first and second portions from each other.

Example 29. The method of any one of Examples 16 to 28, further comprising charging the medical device with the provided power.

Example 30. The method of any one of Examples 16 to 29, wherein the medical device is a leadless pacemaker.

Example 31. A leadless pacemaker comprising:
a metallic housing containing a battery and circuitry for generating electrical pulses for stimulating cardiac tissue;
a pacing electrode located at a distal end of the housing, the pacing electrode being in electrical communication with the circuitry; and
a polymeric seal located at a proximal end of the housing, the polymeric seal providing, upon passage therethrough, access to a first portion in the housing, the first portion being configured to provide electrical contact and facilitate charging the battery.

Example 32. The leadless pacemaker of Example 31, wherein the polymeric seal is an unbroken monolith which is configured to admit access to the first portion upon being pierced.

Example 33. The leadless pacemaker of Example 31, wherein the polymeric seal defines a passage which is liquid-tight but will allow a charging shaft to pass through to the first portion without need for piercing or tearing the polymeric seal.

Example 34. The leadless pacemaker of any of Examples 31 to 33, further comprising a second portion positioned in the housing, the second portion being configured to provide electrical contact and facilitate charging the battery, the polymeric seal providing, upon passage therethrough, access to the second portion.

Example 35. The leadless pacemaker of any of Examples 31 to 33, further comprising a second portion positioned on an exterior of the housing, the second portion being configured to provide electrical contact and facilitate charging the battery.

Example 36. The leadless pacemaker of any of Examples 31 to 35, further comprising an attachment member at the proximal end of the housing, the attachment member comprising a neck extending proximally from the housing and a head at a proximal end of the neck, the neck having a smaller diameter than the housing, and the head having a larger diameter than the neck.

Example 37. The leadless pacemaker of Example 36, wherein the polymeric seal makes liquid-tight a passage through the neck to the first portion.

Example 38. The leadless pacemaker of Example 37, wherein the polymeric seal makes liquid-tight a passage through the neck to the second portion.

Example 39. The leadless pacemaker of any of Examples 36 to 38, wherein the polymeric seal forms at least a portion of a proximal end of the head.

Example 40. The leadless pacemaker of any of Examples 31 to 39, wherein the polymeric seal forms a tapered recess which faces proximally from a proximal end of the leadless pacemaker.

Example 41. A system for providing power to the leadless pacemaker of any of Examples 31 to 40, the system comprising:
a charging shaft comprising a first electrode near a distal end thereof, the charging shaft being configured to pass through the polymeric seal and make electrical contact between the first electrode and the first portion to facilitate charging the battery.

Example 42. The system of Example 41, wherein the charging shaft further comprises a second electrode near a distal end thereof, the charging shaft being configured to make electrical contact, upon passing through the polymeric seal, between the second electrode and the second portion to facilitate charging the battery.

Example 43. The system of Example 41, further comprising:
an elongate shaft comprising a distal end that is configured to be positioned proximate to the leadless pacemaker in the body of the patient;
a hollow receptacle at the distal end of the elongate shaft, wherein the hollow receptacle is configured to receive at least a portion of the leadless pacemaker; and
an external power source configured to be electrically connected to the first electrode to provide power to the medical device.

Example 44. The system of Example 43, further comprising a second electrode configured to couple to the second portion of the medical device, wherein the second electrode is located on the hollow receptacle.

Example 45. A method for providing power to a medical device in a body of a patient, the method comprising:
introducing a distal end of an elongate shaft within the body of the patient;
receiving at least a portion of the medical device within the body of the patient within a hollow receptacle at the distal end of the elongate shaft;
advancing a distal tip of a charging shaft through a polymeric seal at a proximal end of the medical device;
coupling a first electrode to a first portion of the medical device disposed beyond the polymeric seal, wherein the first electrode is located at or near the distal tip of the charging shaft;
coupling a second electrode to a second portion of the medical device; and
providing power to the medical device via the first and second electrodes.

Example 46. The system of any of Examples 1 to 15, wherein:
the medical device comprises a polymeric seal disposed at a proximal end of the medical device, and the second portion of the medical device is positioned beyond the polymeric seal;
the second electrode is disposed at or near a distal end of a charging shaft; and
the charging shaft is configured to pass through the polymeric seal to couple to the
second portion of the medical device after passing through the polymeric seal.

Example 47. The system of Example 46, wherein the polymeric seal is an unbroken monolith which is configured to admit access to the second portion upon being pierced.

Example 48. The system of Example 46, wherein the polymeric seal defines a passage which is liquid-tight but will allow the charging shaft to pass through to the second portion without need for piercing or tearing the polymeric seal.

### Conclusion

Although many of the embodiments are described above with respect to systems, devices, and methods for delivering power to an implantable device in a patient’s heart, the technology is applicable to other applications and/or other approaches, such as deliver of power to other types of implantable devices. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1–9B.

The embodiments of the present technology can be implemented, at least in part, in hardware, software, firmware or any combination thereof. For example, various embodiments can be implemented within one or more processors, including one or more microprocessors, DSPs, ASICs, FPGAs, or any other equivalent integrated or discrete logic circuitry, as well as any combinations of such components, embodied in programmers (e.g., physician or patient programmers), stimulators, or other devices. The terms “processor” or “processing circuitry” may generally refer to any of the foregoing logic circuitry, alone or in combination with other logic circuitry, or any other equivalent circuitry.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A system for providing power to a medical device in a body of a patient, the system comprising:
an elongate shaft comprising a distal end that is configured to be positioned proximate to the medical device in the body of the patient;
a hollow receptacle at the distal end of the elongate shaft, wherein the hollow receptacle is configured to receive at least a portion of the medical device;
a first electrode configured to couple to a first portion of the medical device, wherein the first electrode is located on the hollow receptacle;
a second electrode configured to couple to a second portion of the medical device; and
an external power source configured to be electrically connected to the first and second electrodes to provide power to the medical device.

2. The system of claim 1, wherein the first electrode is positioned at a first location on the hollow receptacle, and wherein the second electrode is positioned at a second location on the hollow receptacle different from the first location.

3. The system of claim 2, wherein the first location and second locations are spaced apart from each other along a longitudinal axis of the hollow receptacle.

4. The system of claim 1, further comprising a capture element configured to releasably couple to the second portion of the medical device, wherein the second electrode is located on the capture element.

5. The system of claim 4, wherein the elongate shaft comprises a lumen, and wherein the capture element is positioned at least partially within the lumen.

6. The system of claim 1, wherein the first electrode is configured to directly contact the first portion of the medical device, and wherein the second electrode is configured to directly contact the second portion of the medical device.

7. The system of claim 1, wherein the hollow receptacle is configured to extend around a circumference or perimeter of the medical device.

8. The system of claim 1, further comprising a spring element, wherein the spring element is configured to bias one or more of the first electrode or the second electrode into contact with the first portion or the second portion, respectively, of the medical device.

9. The system of claim 1, further comprising a cutting element configured to remove tissue overgrowth on the medical device.

10. The system of claim 1, wherein the first and second portions are part of a housing of the medical device.

11. The system of claim 10, wherein the housing comprises an insulating region configured to electrically isolate the first and second portions from each other.

12. The system of claim 1, wherein the medical device is a leadless pacemaker.

13. The system of claim 1, wherein:
the medical device comprises a polymeric seal disposed at a proximal end of the medical device, and the second portion of the medical device is positioned beyond the polymeric seal;
the second electrode is disposed at or near a distal end of a charging shaft; and
the charging shaft is configured to pass through the polymeric seal to couple to the second portion of the medical device after passing through the polymeric seal.

14. The system of claim 13, wherein the polymeric seal is an unbroken monolith which is configured to admit access to the second portion upon being pierced.

15. The system of claim 13, wherein the polymeric seal defines a passage which is liquid-tight but will allow the charging shaft to pass through to the second portion without need for piercing or tearing the polymeric seal.

16. A leadless pacemaker comprising:
a metallic housing containing a battery and circuitry for generating electrical pulses for stimulating cardiac tissue;
a pacing electrode located at a distal end of the housing, the pacing electrode being in electrical communication with the circuitry; and
a polymeric seal located at a proximal end of the housing, the polymeric seal providing, upon passage therethrough, access to a first portion in the housing, the first portion being configured to provide electrical contact and facilitate charging the battery.

17. The leadless pacemaker of claim 16, wherein the polymeric seal is an unbroken monolith which is configured to admit access to the first portion upon being pierced.

18. The leadless pacemaker of claim 16, wherein the polymeric seal defines a passage which is liquid-tight but will allow a charging shaft to pass through to the first portion without need for piercing or tearing the polymeric seal.

19. The leadless pacemaker of claim 16, further comprising an attachment member at the proximal end of the housing, the attachment member comprising a neck extending proximally from the housing and a head at a proximal end of the neck, the neck having a smaller diameter than the housing, and the head having a larger diameter than the neck.

20. The leadless pacemaker of claim 19, wherein the polymeric seal makes liquid-tight a passage through the neck to the first portion.
